(19) United States
(12) Patent Application Publication
(10) Pub. No.: US 2023/0255552 A1
(43) Pub. Date: Aug. 17, 2023

(54) SYSTEMS AND METHODS FOR JOINT HEALTH ASSESSMENT

(71) Applicant: Georgia Tech Research Corporation, Atlanta, GA (US)

(72) Inventors: Omer Inan, Atlanta, GA (US); Samer Mabrouck, Atlanta, GA (US)

(21) Appl. No.: 18/012,451

(22) PCT Filed: Jun. 25, 2021
(86) PCT No.: PCT/US2021/039069

§ 371 Date: Dec. 22, 2022

Related U.S. Application Data
(60) Provisional application No. 63/044,508, filed on Jun. 26, 2020.

(51) Int. Cl. A61B 5/00; A61B 5/0537; A61B 5/11

(52) U.S. Cl. CPC A61B 5/4595; A61B 5/0537; A61B 5/4878; A61B 5/1114; A61B 5/1126; A61B 5/1121; A61B 5/6829; A61B 5/0002; A61B 2562/0219; A61B 2562/06

(57) ABSTRACT

An exemplary embodiment of the present disclosure provides a system for assessing joint health comprising a joint sensor configured to measure at least one non-acoustic characteristic of a joint during movement; a bioimpedance sensor configured to measure bioimpedance of a joint structure exposed to electrical current at a plurality of frequencies; a processor; and a memory, the memory comprising instructions that, when executed by the processor, cause the processor to provide an assessment of joint health through interpretation of measurements from the joint sensor and the bioimpedance sensor.

### CROSS-REFERENCE TO RELATED APPLICATIONS

This application claims the benefit of U.S. Provisional Application Ser. No. 63/044,508, filed on Jun. 26, 2020, which is incorporated herein by reference in its entirety as if fully set forth below.

### GOVERNMENT LICENSE RIGHTS

This disclosure was made with government support under Award No. N66001-19-2-4002 awarded by DARPA/Naval Information Warfare Center and Award No. NIH R01EB023808 awarded by the National Institutes of Health. The government has certain rights in the disclosure.

### FIELD OF THE DISCLOSURE

The present disclosure relates generally to health systems and methods and more particularly to a wearable system and method that assesses the health status of a user's joint and informs the user and/or caregiver of the results.

### BACKGROUND

Musculoskeletal injuries, like a sprained joint, are very common. For example, a total of 23,000 ankle sprains occur per day in the United States—91% of which are lateral ankle sprains—making it the most common sports-related musculoskeletal injury. After the first sprain, a patient is much more likely to reinjure the ankle and some patients may even experience long-term disability. An ankle sprain is initially evaluated based on the presence and level of edema and limitations to the joint's range of motion. Both measures are qualitative, subjective, and rely on a healthcare worker's expertise. Imaging studies are often used to diagnose the injury by revealing structural abnormalities or ligament tears, but these studies are expensive, time-consuming, may expose the patient to radiation, and require an expert to interpret the findings. Physical examination alone has a diagnostic sensitivity of 96% and specificity of 84%.

Musculoskeletal injuries have characteristically long recovery times. After diagnosis, with appropriate medical interventions, a patient enters a period of recovery and rehabilitation. During this rehabilitative period, repeat clinical visits or imaging studies are impractical. Ideally, wearable technologies could be used to provide constant feedback to patients during this period. However, the only currently available technologies for quantifying ankle health status focus on the joint's range of motion, most commonly assessed using inertial measurements. These range of motion measurements do not fully capture the physiological changes occurring in a healing joint. Additional technologies need to be developed to provide precise, actionable feedback to the patient for optimizing their rehabilitation without relying solely on clinician input.

Therefore, what is needed is a method and system for assessing joint health capable of detecting variations in joint health, such as edema or structural integrity, and providing feedback to user's and/or caregivers during rehabilitation and activity, when it is most needed to avoid reinjury.

### SUMMARY

The present disclosure relates to health systems and methods. The disclosed technology includes a system for assessing joint health. The system for assessing joint health can include a joint sensor, a bioimpedance sensor, a processor, and a memory. The joint sensor can be configured to measure at least one non-acoustic characteristic of a joint. The bioimpedance sensor can be configured to measure bioimpedance of the joint exposed to electrical current at a plurality of frequencies. The memory can comprise instructions that, when executed by the processor, cause the processor to provide an assessment of joint health through interpretation of measurements from the joint sensor and the bioimpedance sensor.

The assessment of joint health can differentiate between a healthy joint and an injured joint.

The assessment of joint health can occur in real-time during movement of the joint.

The assessment of joint health can comprise a detection of changes in edema in the joint during movement of the joint.

The assessment of joint health can comprise a detection of changes in tissue integrity during movement of the joint.

The bioimpedance sensor can be configured to sense reactance at the plurality of frequencies.

The bioimpedance sensor can be configured to sense resistance at the plurality of frequencies.

The memory can comprise instructions that, when executed by the processor, cause the processor to compare changes in bioimpedance at the plurality of frequencies during movement of the joint to determine a ratio of the changes in bioimpedance.

The memory can comprise instructions that, when executed by the processor, cause the processor to provide the assessment of joint health based at least in part on the ratio of the changes in bioimpedance.

The joint sensor can measure a gait cycle.

The memory can comprise instructions that, when executed by the processor, cause the processor to provide a window of time representing each step of the gait cycle at least in part from measuring a heel strike.

The bioimpedance sensor can be configured to sense a range per step of bioimpedance at the plurality of frequencies.

The memory can comprise instructions that, when executed by the processor, cause the processor to detect changes in joint edema at least in part by taking a ratio of the range per step of bioimpedance at the plurality of frequencies.

The joint sensor can measure a walking session.

The bioimpedance sensor can be configured to sense a range per walking session of bioimpedance at the plurality of frequencies.

The memory can comprise instructions that, when executed by the processor, cause the processor to detect changes in tissue integrity in the joint at least in part by taking a ratio of the range per walking session of bioimpedance at the plurality of frequencies.

The joint sensor can be a kinematic sensor configured for sensing characteristics related to joint movement.

The joint sensor can comprise one or more inertial measurement units.

The joint sensor can be configured to measure at least angular velocity at the joint.

The bioimpedance sensor can be configured to sense characteristics related to bioimpedance of the joint when the angular velocity equals zero during movement.

The plurality of frequencies can comprise a first frequency and a second frequency.

The bioimpedance sensor can deliver a first current at the first frequency such that the first current can propagate through extra-cellular fluid.

The bioimpedance sensor can deliver a second current at the second frequency such that it can propagate through intra-cellular fluid and extra-cellular fluid.

The first frequency can be 1-50 kHz.

The second frequency can be 50-1000 kHz.

The joint sensor can comprise a first wearable sensor for placement proximate the joint. The bioimpedance sensor can comprise a second wearable sensor for placement proximate the joint.

The system for assessing joint health can comprise an output capable of providing an indication of joint health to a user of the system.

The system for assessing joint health can comprise a wireless communicator.

The joint can be an ankle.

The memory can comprise instructions that, when executed by the processor, cause the processor to perform a full frequency sweep analysis when the joint is not in movement.

The disclosed technology includes a system for assessing joint health. The system for assessing joint health can include a joint sensor, a bioimpedance sensor, a processor, and a memory. The joint sensor can be configured to measure at least one non-acoustic characteristic of a joint. The bioimpedance sensor can be configured to measure bioimpedance of the joint exposed to electrical current at a plurality of frequencies. The memory can comprise instructions that, when executed by the processor, cause the processor to compare changes in bioimpedance at the plurality of frequencies during movement of the joint to determine a ratio of the changes in bioimpedance. The memory can comprise instructions that, when executed by the processor, cause the processor to provide an assessment of joint health based at least in part on the ratio of the changes in bioimpedance.

The disclosed technology includes a method for assessing joint health. The method can include measuring, with a joint sensor, at least one non-acoustic characteristic of a joint. The method can include measuring, with a bioimpedance sensor, bioimpedance of the joint exposed to electrical current at a plurality of frequencies. The method can include providing, with a memory and a processor, an assessment of joint health through interpretation of measurements from the joint sensor and the bioimpedance sensor.

The assessment of joint health can differentiate between a healthy joint and an injured joint.

The assessment of joint health can occur in real-time during movement of the joint.

The assessment of joint health can comprise a detection of changes in edema in the joint during movement of the joint.

The assessment of joint health can comprise a detection of changes in tissue integrity during movement of the joint.

The bioimpedance sensor can be configured to sense reactance at the plurality of frequencies.

The bioimpedance sensor can be configured to sense resistance at the plurality of frequencies.

The method can comprise comparing changes in bioimpedance at the plurality of frequencies during movement of the joint to determine a ratio of the changes in bioimpedance.

The method can comprise providing the assessment of joint health based at least in part on the ratio of the changes in bioimpedance.

The joint sensor can measure a gait cycle.

The method can comprise providing a window of time representing each step of the gait cycle at least in part from measuring a heel strike.

The bioimpedance sensor can be configured to sense a range per step of bioimpedance at the plurality of frequencies.

The method can comprise detecting changes in joint edema at least in part by taking a ratio of the range per step of bioimpedance at the plurality of frequencies.

The joint sensor can measure a walking session.

The bioimpedance sensor can be configured to sense a range per walking session of bioimpedance at the plurality of frequencies.

The method can comprise detecting changes in tissue integrity in the joint at least in part by taking a ratio of the range per walking session of bioimpedance at the plurality of frequencies.

The joint sensor can be a kinematic sensor configured for sensing characteristics related to joint movement.

The joint sensor can comprise one or more inertial measurement units.

The joint sensor can be configured to measure at least angular velocity at the joint.

The bioimpedance sensor can be configured to sense characteristics related to bioimpedance of the joint when the angular velocity equals zero during movement.

The plurality of frequencies can comprise a first frequency and a second frequency.

The bioimpedance sensor can deliver a first current at the first frequency such that the first current can propagate through extra-cellular fluid.

The bioimpedance sensor can deliver a second current at the second frequency such that it can propagate through intra-cellular fluid and extra-cellular fluid.

The first frequency can be 1-50 kHz.

The second frequency can be 50-1000 kHz.

The joint sensor can comprise a first wearable sensor for placement proximate the joint. The bioimpedance sensor can comprise a second wearable sensor for placement proximate the joint.

The method can comprise an output capable of providing an indication of joint health to a user of the system.

The method can comprise a wireless communicator.

The joint can be an ankle.

The method can comprise performing a full frequency sweep analysis when the joint is not in movement.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features can also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a photo of an example system for assessing joint health, in accordance with the present disclosure.

FIG. 1B provides a drawing of an example system for assessing joint health, in accordance with the present disclosure.

FIG. 2 provides diagrams and graphs of ankle edema tracking methods, in accordance with the present disclosure.

FIG. 3(a) provides photo of a saline injection into cadaver ankle, in accordance with the present disclosure. FIG. 3(b) provides an example system for assessing joint health, in accordance with the present disclosure. FIG. 3(c) provides a block diagram of an example system for assessing joint health, in accordance with the present disclosure. FIG. 3(d) provides a block diagram of an example voltage controlled current source system, in accordance with the present disclosure.

FIG. 4 provides a diagram of calibration methods, in accordance with the present disclosure.

FIG. 5(a) provides photos of ankle positions used in experimentation, in accordance with the present disclosure. FIG. 5(b) provides experimentally measured changes in edema, in accordance with the present disclosure. FIG. 5(c) provides experimentally measured changes in edema, in accordance with the present disclosure.

FIG. 6(a) provides a scatter plot of intra-subject variability, in accordance with the present disclosure. FIG. 6(b) provides a box plot of inter-subject variability, in accordance with the present disclosure.

FIG. 7 provides a graph of experimentally measured resistance, in accordance with the present disclosure.

FIG. 8(a) provides an illustration of an ankle joint, in accordance with the present disclosure. FIG. 8(b) provides an illustration of a blood vessel, in accordance with the present disclosure. FIG. 8(c) provides an illustrations of muscle fibers, in accordance with the present disclosure. FIG. 8(d) provides an illustration of a blood vessel, in accordance with the present disclosure. FIG. 8(e) provides an illustrations of muscle fibers, in accordance with the present disclosure. FIG. 8(f) provides experimentally measured reactance, in accordance with the present disclosure.

FIG. 9(a) provides a photo of an example system for assessing joint health, in accordance with the present disclosure. FIG. 9(b) provides experimentally measured, angular velocity, acceleration, and reactance, in accordance with the present disclosure. FIG. 9(c) provides experimentally measured, angular velocity, acceleration, and reactance, in accordance with the present disclosure. FIG. 9(d) provides an analysis method to detect edema and structural integrity, in accordance with the present disclosure.

FIG. 10(a) provides photos of an example system for assessing joint health, in accordance with the present disclosure. FIG. 10(b) provides a recording protocol timeline, in accordance with the present disclosure. FIG. 10(c) provides an experimental positional protocol, in accordance with the present disclosure.

FIG. 11(a) provides experimentally measured reactance, in accordance with the present disclosure. FIG. 11(b) provides experimentally measured reactance, in accordance with the present disclosure. FIG. 11(c) provides a correlation method, in accordance with the present disclosure.

FIG. 12(a) provides a graph of experimentally measured change in reactance per step, in accordance with the present disclosure. FIG. 12(b) provides a scatter plot of experimentally measured change in reactance per step, in accordance with the present disclosure. FIG. 12(c) provides a graph of experimentally measured change in reactance per step, in accordance with the present disclosure. FIG. 12(d) provides a graph of experimentally measured change in reactance per walking session, in accordance with the present disclosure. FIG. 12(e) provides a scatter plot of experimentally measured change in reactance per walking session, in accordance with the present disclosure.

FIG. 13 provides a graph of bioimpedance spectroscopy and estimated impedance, in accordance with the present disclosure.

FIG. 14 provides a flow chart illustrating an example method for assessing joint health, in accordance with the present disclosure.

### DETAILED DESCRIPTION

Throughout this disclosure we describe a system for assessing joint health. For example, a system for assessing joint health that can be wearable system with a kinematic sensor and bioimpedance sensor for providing real-time assessment of joint health. As such, the system can assess health of a joint such as joint edema and structural integrity.

While the disclosed technology is described throughout this disclosure in relation to a system for assessing joint health, those having skill in the art will recognize that the disclosed technology is not so limited and can be applicable to other scenarios and applications. For example, it is contemplated that the disclosed technology can be applicable to any musculoskeletal health, including, but not limited to, a pulled muscle or broken bone.

Some implementations of the disclosed technology will be described more fully with reference to the accompanying drawings. This disclosed technology may, however, be embodied in many different forms and should not be construed as limited to the implementations set forth herein. The components described hereinafter as making up various elements of the disclosed technology are intended to be illustrative and not restrictive. Indeed, it is to be understood that other examples are contemplated. Many suitable components that would perform the same or similar functions as components described herein are intended to be embraced within the scope of the disclosed electronic devices and methods. Such other components not described herein may include, but are not limited to, for example, components developed after development of the disclosed technology.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified. Further, it is contemplated that the disclosed methods and processes can include, but do not necessarily include, all steps discussed herein. That is, methods and processes in accordance with the disclosed technology can include some of the disclosed while omitting others.

Throughout the specification and the claims, the following terms take at least the meanings explicitly associated herein, unless otherwise indicated. The term “or” is intended to mean an inclusive “or.” Further, the terms “a,” “an,” and “the” are intended to mean one or more unless specified otherwise or clear from the context to be directed to a singular form. By “comprising,” “containing,” or “including” it is meant that at least the named element, or method step is present in article or method, but does not exclude the presence of other elements or method steps, even if the other such elements or method steps have the same function as what is named.

As used herein, unless otherwise specified, the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

Although the disclosed technology may be described herein with respect to various systems and methods, it is contemplated that embodiments or implementations of the disclosed technology with identical or substantially similar features may alternatively be implemented as methods or systems. For example, any aspects, elements, features, or the like described herein with respect to a method can be equally attributable to a system. As another example, any aspects, elements, features, or the like described herein with respect to a system can be equally attributable to a method.

Reference will now be made in detail to examples of the disclosed technology, examples of which are illustrated in the accompanying drawings and disclosed herein. Wherever convenient, the same reference numbers will be used throughout the drawings to refer to the same or like parts.

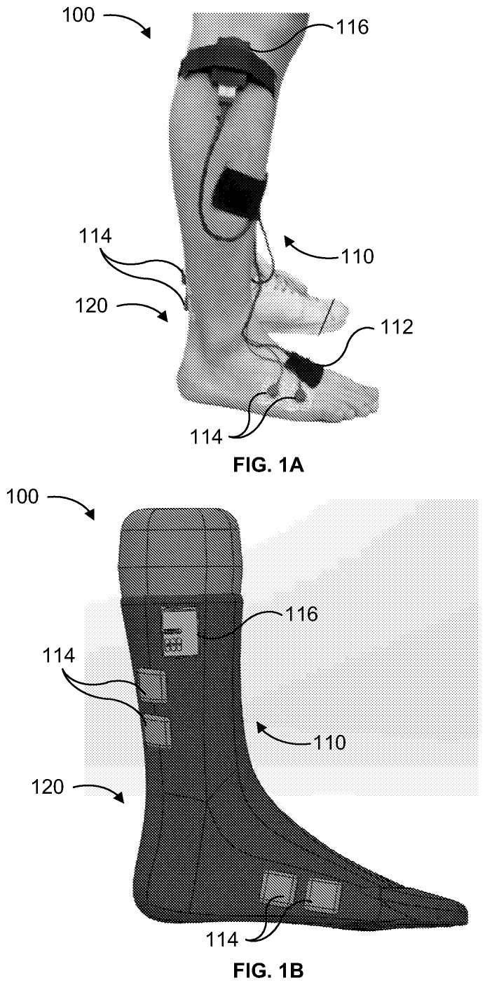

Referring now to the drawings, in which like numerals represent like elements, examples of the present disclosure are herein described. As will be described in greater detail, the present disclosure can include a system and method for assessing joint health. To provide a background of the system described in the present disclosure, components of the system for assessing joint health is shown in FIG. 1 and will be discussed first.

To facilitate an understanding of the principles and features of the present disclosure, various examples of the disclosed technology are explained herein. The components, steps, and materials described herein as making up various elements of the disclosed technology are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used herein, unless otherwise noted, the term “joint health” refers to the health of the entire musculoskeletal system of a joint and surrounding a joint. For example, the health of the bone, muscles, and soft tissue.

As shown in FIGS. 1A and 1B, the disclosed technology includes a system for assessing joint health 100. The system 100 can include a wearable device 110. The wearable device 110 can be a device worn on a person and proximate a joint 120. For example, as illustrated in FIG. 1B, the wearable device 110 can be sleeve or sock with the components embedded therein.

The wearable device 110 can include one or more sensors. For example, the wearable device 110 can include a joint sensor 112. Alternatively, or in addition, the wearable device 110 can include a bioimpedance sensor 114. Alternatively, or in addition, the wearable device 110 can include electronics 116.

Alternatively, or in addition, the wearable device 110 can include a processor and a memory. For example, the wearable device can include CPU, microprocessor, and the like. The memory can comprise logical instructions that, when executed by the processor, cause the processor to carry out one of more of the functions disclosed herein. Alternatively, or in addition, the wearable device 110 can include a transceiver. For example, the transceiver can receive data from the one or more sensors (e.g., joint sensor 112, bioimpedance sensor 114) and transmit data to a remote device. The wearable device can include a power source. For example, the power source can be a battery for powering the components of the wearable device (e.g., joint sensor 112, bioimpedance sensor 114, processor, transceiver).

The electronics 116 can include electronic components of the system 100. For example, the electronics 116 can include the processor, transceiver, power source, and sensor circuit. Alternatively, or in addition, the electronics 116 can include one or more sensors. For example, the electronics 116 can include the joint sensor 112 (e.g., one or more inertial measurement units).

The joint sensor 112 can be configured to measure at least one non-acoustic characteristic of a joint. For example, the joint sensor 112 can be a kinematic sensor configured for sensing characteristics related to joint movement (e.g., angular velocity). The joint sensor 112 can include one or more inertial measurement units.

The bioimpedance sensor 114 can be configured to measure bioimpedance at a joint 120. For example, the bioimpedance sensor can include a source of current and a receiver. The Bioimpedance sensor can measure the opposition to electric current through the body. For example, the impedance across a joint 120. The bioimpedance sensor 114 can measure electrical resistance. Alternatively, or in addition, the bioimpedance sensor 114 can measure reactance. The bioimpedance sensor 114 can measure bioimpedance at a plurality of frequencies. For example, the bioimpedance sensor 114 can measure bioimpedance at a low frequency and a high frequency. The low frequency can be a frequency such that the current at the first frequency can propagate through extra-cellular fluid. The high frequency can be a frequency a frequency such that the current at the second frequency can propagate through intra-cellular fluid and extracellular fluid. For example, the low frequency can be a frequency from 1-50 kHz and the high frequency can be a frequency from 50-1000 kHz.

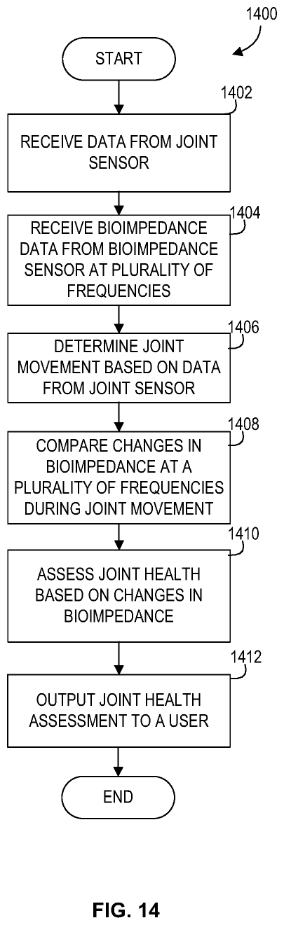

The disclosed technology includes methods for assessing joint health, such as method 1400, which is illustrated in FIG. 14. Method 1400 and/or any other method described herein can be performed by a controller or computer.

The method 1400 can include receiving 1402 data from a joint sensor (e.g., a kinematic sensor, inertial measurement unit). The data from the joint sensor can relate to at least one non-acoustic characteristic of a joint during movement. For example, the joint sensor can measure the angular velocity at the joint.

The method 1400 can include receiving 1404 data from a bioimpedance sensor. The bioimpedance data can be at a plurality of frequencies. For example, the bioimpedance sensor can measure bioimpedance at a low frequency and a high frequency. The bioimpedance sensor can measure changes in reactance across a joint. The bioimpedance sensor can measure changes in resistance across a joint.

The method 1400 can include determining 1406, based on the joint sensor data, joint movement. For example, a user's gait cycle can be determined based on data received from the joint sensor. The gait cycle can be determined bases at least in part from measuring a heel strike from data from the joint sensor. Alternatively, or in addition, a walking session can be determined based on data from the joint sensor.

The method 1400 can include comparing 1408, based on the bioimpedance sensor data, changes in bioimpedance at a plurality of frequencies. For example, comparing the changes in bioimpedance per step based on each step of the user's gait cycle, as determined based on the joint sensor data. Alternatively, or in addition, comparing changes in bioimpedance per walking session, based on the user's walking session, as determined based on the joint sensor data.

The method 1400 can include assessing 1410, based on changes in bioimpedance, joint health. For example, edema in the joint can be determined based on changes in bioimpedance (e.g., ratio of the range per step of bioimpedance at a plurality of frequencies). Alternatively, or in addition, tissue integrity in the joint can be determined based on changed in bioimpedance (e.g., ration of the range per walking session of bioimpedance at a plurality of frequencies).

The method 1400 can include outputting 1412 the joint health assessment to a user. For example, the joint health assessment can be sent to a connected device (e.g., smart phone, tablet, computer). Alternatively, or in addition, the joint health assessment can be displayed on a joint health assessment device (e.g., a wearable device).

The following examples further illustrate aspects of the present disclosure. However, they are in no way a limitation of the teachings or disclosure of the present disclosure as set forth herein.

### EXAMPLES

This disclosure presents a robust methodology for tracking ankle edema longitudinally based on bioimpedance spectroscopy (BIS). Methods: We designed a miniaturized BIS measurement system and employed a novel calibration method that enables accurate, high resolution measurements with substantially lower power consumption than conventional approaches. Using this state-of-the-art wearable BIS measurement system, we developed a differential measurement technique for robust assessment of ankle edema. This technique addresses many of the major challenges in longitudinal BIS-based edema assessment, including day-to-day variability in electrode placement, positional/postural variability, and inter-subject variability. Results: We first evaluated the hardware in bench-top testing, and determined the error of the bioimpedance measurements to be 0.4Ω for the real components and 0.54Ω for the imaginary components with a resolution of 0.2Ω. We then validated the hardware and differential measurement technique in (1) an ex-vivo, fresh-frozen, cadaveric limb model, and (2) a cohort of 11 human subjects for proof of concept (8 healthy controls and 5 subjects with recently acquired acute unilateral ankle injury). Conclusion: The hardware design, with novel calibration methodology, and differential measurement technique, can enable long-term quantification of ankle edema throughout the course of rehabilitation following acute ankle injuries. Significance: This can lead to better-informed decision making regarding readiness to return to activities and/or tailoring of rehabilitation activities to an individual's changing needs.

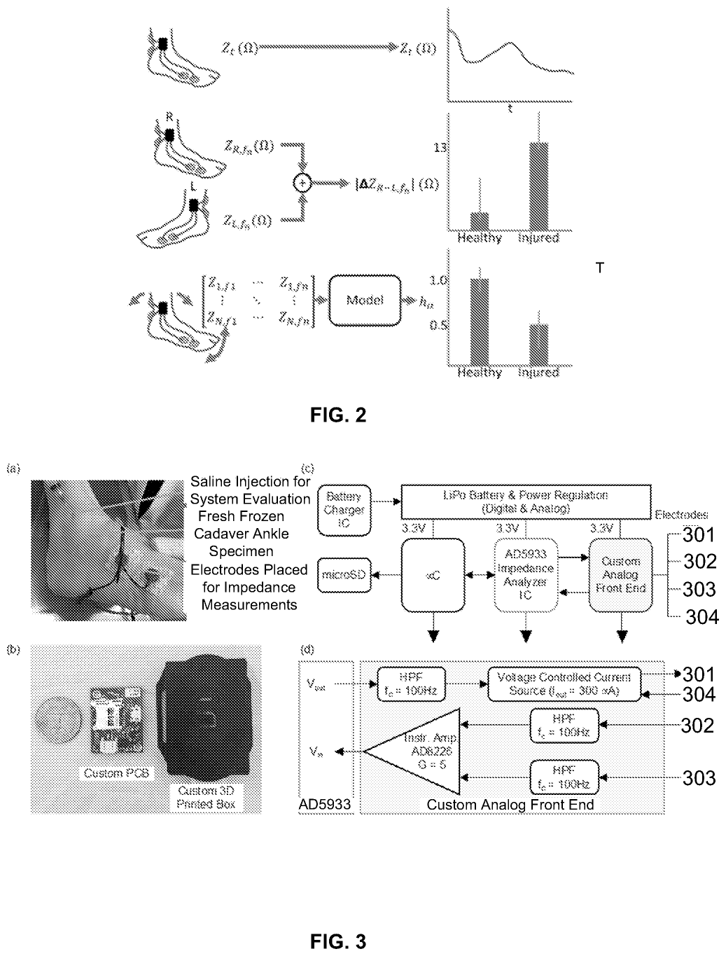

Thus, this disclosure presents a wearable BIS measurement system our group has designed and optimized for low-power, accurate and robust measurement of edema in the ankle. This disclosure includes: (1) an innovative calibration methodology based on physiology-driven principles by leveraging a multi-point linear calibration model based on least squares that allows for both low power and accurate BIS measurement in a wearable form factor; and (2) a differential measurement technique that exploits postural variations in the position of fluid within the joint space to reduce inter- and intra-subject variability in edema quantification and removes the need to compare the affected joint against the contralateral side for normalization. The concept of this differential measurement technique is summarized in FIG. 2. FIG. 2 provides three different ankle edema tracking methods that use BIA (a) by only tracking the ankle impedance longitudinally, (b) by using the contralateral ankle to compare to the injured ankle's impedance to and (c) by comparing the change in impedance at different frequencies due to changes in ankle position Specifically, rather than using the impedance measurement itself for one ankle (top), or computing the difference between the injured and contralateral ankle (middle), our technique involves having the subject move his/her ankle to multiple positions, and examining the manner in which such movement affects the lower and higher frequency components of impedance (bottom). This research would facilitate tracking edema in a recently injured ankle joint, such as acute edema caused by repetitive stress. Tracking acute edema is often challenging for medical professionals as it is induced by certain activities that are difficult to perform in the clinic, such as medium to high intensity workouts. This device would help in tracking acute edema, particularly outside clinical settings such as in fitness centers, recreational facilities, and the workplace (for persons in occupations requiring standing for long periods of time or otherwise active professions); this edema tracking could then enable better decisions regarding rehabilitation.

### Wearable Bis System Design and Characteristics

### A. System Overview

The positioning of the electrodes on the ankle joint is shown in FIG. 3(a) and a photo of the wearable BIS measurement system is shown in FIG. 3(b). The electronic design incorporates discrete components and can include a commercially-available impedance analyzer integrated circuit (IC): AD5933 (Analog Devices, Cambridge, Mass.). The system can be powered by a 500 mAh LiPo battery with a battery charger on board, enclosed in a 5.2×3.8×1.8 cm box.

The system is divided into a digital and an analog block where each block is powered through a separate voltage regulator. The digital block consists of an ultra-low power microcontroller (SAMD21, Atmel, San Jose, Calif.) that has multiple serial interfaces for communication, and a Secure Digital (SD) card for data logging. The analog block consists of the AD5933 coupled to an analog front end (AFE) consisting of a high bandwidth, low power and low noise voltage controlled current source (VCCS) and an instrumentation amplifier to facilitate four-electrode measurement. This analog front end can be included for two reasons: (1) the AD5933 IC is designed for two-electrode impedance analysis, while for BIS measurements a full four-electrode measurement is necessary to remove the skin-electrode interface impedance component; and (2) the IC delivers a voltage and expects a corresponding current measurement, while the safety guidelines in IEC 60601-1-11 outline that the current delivered to the body must be limited.

The VCCS topology is a single op-amp with the load in the loop. The injected current in our system is limited to 280 μA rms with no direct current (DC) component. The designed VCCS has a dynamic range of 4 kΩ, which exceeds the typical ankle impedance of 180 Ω along with the skin-electrode interface impedance of approximately 330 Ω measured at 5 kHz for the Ag/AgCl gel-based electrodes used in this study.

The output current from this VCCS, Iₒᵤₜ, excites the body that enables current to flow through extracellular and intracellular fluid paths. The current is injected through electrode 301 and collected through electrode 304, as shown in FIG. 3(d). The frequency of this current is swept discretely by issuing an I2C command to the AD5933. At each discrete frequency the potential difference across electrodes 302 and 303 is measured by an instrumentational amplifier (AD8226). The output of this amplifier is used by the DSP core of the AD5933 to calculate real and imaginary 16-bit values that can be used to identify the impedance of the interrogated tissue volume. The frequency sweep of 5 kHz to 100 kHz is completed in 3.5 sec with 371 increments of 256 Hz.

The separation of the digital and analog components improves the signal integrity and lowers power consumption as the shutdown pin on the analog regulator is controlled by the microcontroller through by a GPIO pin leading to a complete shutdown of the analog block of the system. Three LEDs are used to notify the user for system errors, low battery and the charging status. The SD card shield is a push IN/push OUT shield for ease of use. The system is charged through a 5V 2A DC μUSB charger.

### B. Calibration

### 1) Overview of Existing Methods for AD5933 Calibration and Challenges

We designed and implemented a calibration strategy to maximize safety and accuracy of the BIS system with minimal power consumption and size. In conventional impedance analysis operations, the AD5933 requires a simple calibration process that maps the 16-bit real and imaginary outputs to the real and imaginary values of the impedance being measured. The calibration process requires a single step of measuring a resistive load with a known admittance and calculating the gain factor and phase shift required to map the 16-bit raw values to the actual load impedance. The IC has been often used in literature for bioimpedance analysis where a complex analog front end that has not been optimized for power efficiency is used to enable 4-electrode measurements. The main reason behind the complexity of the AFE is to facilitate the use of the conventional single-point calibration method.

Unfortunately, these AFE designs in the existing literature are not amenable for use in a wearable BIS system as desired for the application of ankle edema quantification at home. Specifically, these existing designs require the use of a current-to-voltage conversion block, such as an opto-isolator, to allow the voltage measurement output from the instrumentation amplifier to be converted to a current value as expected by the AD5933 input. Such a block requires substantial power for operation, and typically requires larger ICs that are not suitable for miniaturized designs. Accordingly, we decided to simplify the AFE design, alleviating the requirement for such a current-to-voltage conversion step, and compensated via a more complex calibration methodology.

### 2) Linear Regression Based Multi-Point Calibration

Overview of Calibration Methodology: Our calibration methodology can use multiple impedance loads coupled with a multivariate linear regression algorithm to allow for the outputted 16-bit raw values generated by the IC to be mapped to accurate real and imaginary impedance values. Additionally, rather than using a simple resistive load, we used the standard 2R1C model for biological tissue to allow for this mapping operation to be accurate across the full range of measured frequencies. Specifically, our method can be described as follows: Let Q be a N×3 matrix combining the 16-bit real, re_{f}, and imaginary, im_{f}, values from the AD5933 for N-loads measured at frequency f.

$${Q}_{f} = [ \begin{matrix}{re}_{f} [ 1 ] & {im}_{f} [ 1 ] & 1 \\ & ⋮ & \\ {re}_{f} [ N ] & {im}_{f} [ N ] & 1\end{matrix} ] \qquad ( 1 )$$

Where Z_{Re,f} and Z_{im,f} are the real and imaginary components of the impedance measured at frequency f mapped from re_{f} and im_{f} using the coefficients in C_{Re,f} and C_{im,f}, respectively, as in (2) and (4). Appendix I further explains the details of applying this model to our system.

$${Z}_{R e , f} = {Q}_{f} \cdot {C}_{R e , f} \qquad ( 2 )$$

$$where$$

$${Z}_{R e , f} = [ \begin{matrix}{Z}_{R e , f} [ 1 ] \\ ⋮ \\ {Z}_{R e , f} [ N ]\end{matrix} ] , {C}_{R e , f} = [ \begin{matrix}{α}_{R e , f} \\ {β}_{R e , f} \\ {γ}_{R e , f}\end{matrix} ] \qquad ( 3 )$$

$${Z}_{I m , f} = {Q}_{f} \cdot {C}_{I m , f} \qquad ( 4 )$$

$$where$$

$${Z}_{I m , f} = [ \begin{matrix}{Z}_{I m , f} [ 1 ] \\ ⋮ \\ {Z}_{I m , f} [ N ]\end{matrix} ] , {C}_{I m , f} = [ \begin{matrix}{α}_{I m , f} \\ {β}_{I m , f} \\ {γ}_{I m , f}\end{matrix} ] \qquad ( 5 )$$

While other groups have used multi-load calibration, such approaches have always been limited to a maximum of two loads (for a linear model) or three loads (for a quadratic model) and a subsequent direct estimation of slope and y-intercept (for linear) and quadratic terms. These approaches are thus sensitive to errors in each individual measurement used for calibration. We use multiple loads (8) for a linear model and combined the information from these loads using least-squares based linear regression. By doing so, we are able to leverage the noise/error reduction properties of linear regression that are well known.

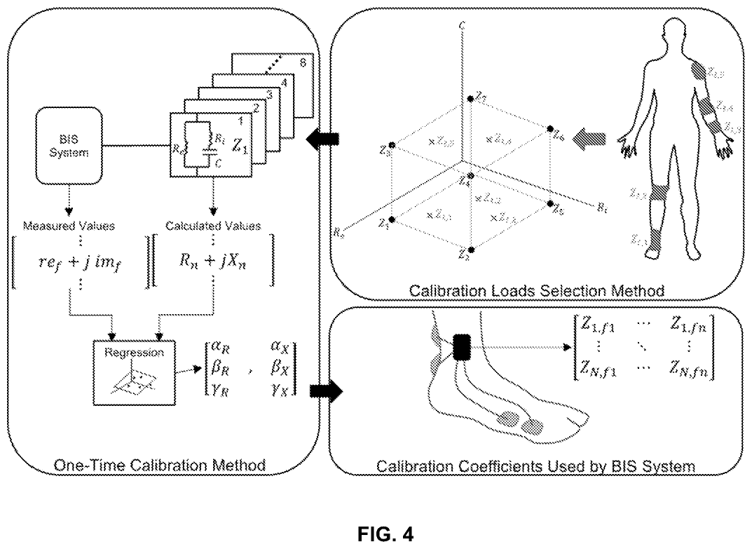

Optimizing the Loads for Calibration: We confined the space of impedance measurements spanned by these three values—2R1C, i.e., two resistors and one capacitor [R_{c}, Rᵢ, C]— to a range that is physiologically meaningful for joint edema measurements. Specifically, we combined values from the existing literature with measured values using commercially available gold standard bioimpedance hardware (SF87, Impedimed, Australia) to form the space of calibration values required. We also investigated the number of loads required to optimize accuracy in impedance measurements with this calibration, and found that N=8, with the particular 2R1C impedance values matching the vertices of the three-dimensional physiologically meaningful impedance space for joint edema provided the lowest error. Investigating the optimal combination of calibration impedances, we varied (1) the span of the combination of calibration impedances in 2R1C space and (2) the number, N, of calibration impedances used in the combination. We tested three different combinations that had the following spans: [R_{c}], [R_{c}, Rᵢ] and [R_{c}, Rᵢ, C]. For each of these combinations we also varied the number of calibration impedances in this span. We found that increasing the number of impedances only reduced the random error in measurements. We also found that the measurement error of an unknown impedance heavily depends on the distance in the 3D space between the measured impedance and the span of the calibration impedances. Hence, we calibrated the system with the impedances in Table III (Appendix II), as they span the linear space up until our dynamic range encompassing the physiologically meaningful impedances as in FIG. 4, ensuring accurate measurements for impedance values falling within this space. FIG. 4 provides a calibration methodology diagram. The calibration loads choice with respect to human body tissue impedance from areas of interest. The data acquired from the device along with the actual impedance values are processed using multivariate linear regression. The coefficients are used to measure bioimpedance from the ankle.

### C. BIS System Characterization

We characterized the BIS system by (1) validating our calibration method, and (2) evaluating the hardware both in terms of electronic specifications and usability.

### 1) Validation of Calibration Methodology

To validate the calibration method, we assembled the 2R1C loads from Table IV (Appendix II) that simulate the bioimpedance of the limbs of interest (e.g., knee, ankle, and elbow) using discrete resistors and capacitors measured separately by Agilent's 34410A Digital Multimeter. The values retrieved from Agilent's DMM were used in Python to simulate the 2R1C impedance at the frequency range over which we performed our spectroscopy. From the simulation, we derived the real and imaginary values of the 2R1C impedances at the different frequency points. These real and imaginary values will be used to evaluate the error in measurements from the device presented.

We focused on three key parameters for sensors to evaluate the calibration methodology: accuracy, resolution and drift. To investigate the accuracy of the bioimpedance sensor we performed a BIS measurement on the 2R1C characterization loads in Table IV using the BIS system; the raw 16-bit real and imaginary values retrieved from the AD5933 were used to compare the calibrated outputs to our simulation results. We calculated the mean error across in real and imaginary components of the characterization impedances over all the frequencies measured to be 0.4 ohms and 0.54 ohms respectively.

Next, we tested the resolution by connecting five 0.1-ohm resistors with 1% tolerance in series to the characteristic 2R1C loads in Table IV, removing one resistor at a time. We found that resistance changes of 0.2 ohms at any frequency for any of the 2R1C characterization loads were detectable above the noise floor of the system. Finally, for drift, we measured a 2R1C load using our device for three days continuously and monitored changes in the impedance spectrum. There was no drift in the bioimpedance measurements throughout the experiment, except for a slight variability of 0.0125 Ω in resistance at 5 kHz and 0.1Ω in resistance at 100 kHz due to a temperature variation of 5° C. Table II shows a comparison of our current design to other AD5933-based designs in literature.

TABLE II
AD5933-Based Designs Comparison

| | | | Number of | | | | | Number of |
|---|---|---|---|---|---|---|---|---|
| | Calibration | Hardware | active | Power | Measurement | Reported | Impedance | test |
| Paper | Method | Topology | components | Consumption | Frequencies | Error | Range | impedances |
| Seoane et | Datasheet | 4-electrode | — | — | 5 kHz-100 kHz | 1% (Not | 1.6 kΩ | 5 (only |
| al[1] | method | VCCS + IA + | | | | clear if real | | resistive) |
| | | V to I | | | | or imaginary) | | |
| Margo et | Datasheet | 4-electrode | >8 | — | 5 kHz-100 kHz | 1.3% argument | 10 kΩ | 1 (2R1C) |
| al [5] | method | VCCS + IA + | | | | 2.5% modulus | | |
| | | V to I | | | | | | |
| Breniue | Datasheet | 4-electrode | 3 | — | 1 kHz-100 kHz | ±2% | 9Ω-18 MΩ | 2 (1R and |
| et al[8] | method | IA + V to I | | | | | | 1L) |
| Bogónez- | Datasheet | 4-electrode | 4 | 34 mA-Data | 100 Hz-200 kHz | 2.5% modulus and | 10Ω-1 kΩ | 2 (RC |
| Franco | method | VCCS + | | Acquisition | | 4.5% argument | | Networks) |
| et al[10] | | 2*Buffer + IA | | 0.2 mA-Sleep | | | | |
| | | | | Mode | | | | |
| Thulasi | Datasheet | 2-electrode | 5 | — | 10 Hz to 100 kHz | 4% Magnitude | 5 kΩ-25 kΩ | Complex |
| et al[12] | method | with constant | | | | and 2 degrees | | impedances |
| | | voltage applied | | | | in phase | | |
| Hafid et | Datasheet | 4-electrode | — | — | 1-350 kHz | 3% | 1Ω-1 KΩ | Complex |
| al[14] | method | IEC 60601 | | | | | | impedances |
| | | compliant | | | | | | |
| | | used in [1] | | | | | | |
| Bogónez- | Datasheet | 4-electrode | 4 | 34 mA-Data | 100 Hz-200 kHz | Modulus error | 10Ω-1 kΩ | 1 (RC |
| Franco | method | VCCS + | | Acquisition | | 0.5% to 2.5%, | | network) |
| et al[15] | | 2*Buffcr + IA | | 0.2 mA-Sleep | | Phase error 2% | | |
| | | | | Mode | | to 4.5% | | |
| Mabrouk | Linear | 4-electrode | 2 | 24 mA in Data | 5 kHz to 100 kHz | Absolute Mean | 0Ω-200Ω | 5 (2R1C |
| et. al | regression | (IEC-60601 | | Acquisition- | | error Real 0.4Ω | | impedances |
| (This | | compliant) | | 1.5 mA in | | (0.34%) Imaginary | | characteristic |
| work) | | VCCS + IA | | Sleep mode | | 0.54Ω (1.36%) | | of joints) |

Note that “—” indicates that the information was not provided.

Alternatives of the system design: The resistive and capacitive components used in calibrating and characterizing the system were measured using a DMM. Alternatively, or in addition an approach could be using actual frequency sweeps from a high accuracy impedance analyzer such as the SFB7 (Impedimed, Australia).

### 2) Evaluating the Hardware for Electronic Specifications and Usability

Table I provides a summary of the key electronic specifications and physical dimensions and weight for the hardware. The power consumption is sufficiently low to enable multi-day measurements from a subject wearing the hardware at home. The dynamic range is high enough to allow for gel, adhesive-backed electrodes for multi-day recordings, but cannot currently facilitate dry electrode measurements. The dynamic range (4 kΩ of the VCCS is sufficient to withstand drift in skin-electrode impedance due to electrodes drying out. The noise floor is below the level required to sense edema changes that are physiological meaningful in the joint.

TABLE I
System Properties

| Parameter | Value | |
|---|---|---|
| Power Consumption-Data collection | 24 | mA |
| Power Consumption-Sleep Mode | 1.5 | mA |
| Sampling Rate | 3 | BIS/min |
| Battery life | 111 | hours |
| Size | 5.2 × 3.8 × 1.8 | cm |
| Weight | 32 | g |
| Frequency Range | 5 kHz-100 kHz | |
| Frequency Resolution | 256 | Hz |
| Dynamic Range | 200 | Ω |
| I_{body} | 280 | μAᵣₘₛ |
| Mean Error in R | 0.4 | Ω |
| Mean Error in X | 0.54 | Ω |
| Resolution | 0.2 | Ω |

Differential Measurement Technique: Evaluation in Cadaver Model and in Subjects with Acute Injury

Using the wearable BIS measurement system described in this disclosure, we conducted studies in a cadaver model as well as in human subjects to assess the accuracy and robustness of edema quantification. Conventional EBI based approaches to quantifying edema in a joint hinge on comparing the injured joint against the contralateral side, and are sensitive to small variations in electrode placement among multiple trials for the same subject, or among studies conducted on multiple subjects. We devised a novel differential measurement technique that leverages the multi-frequency impedance monitoring capability of our BIS system to address these two limitations of standard approaches. The following is (1) a brief description of conventional EBI-based edema quantification methods, as well as the challenges associated with these approaches, (2) a description of our proposed differential measurement technique, and (3) results and discussion from our evaluation of this new methodology in a cadaver model and in human subjects with acute ankle injuries.

### A. Conventional EBI-Based Edema Quantification

In conventional EBI-based approaches the impedance of the joint, Zⱼ, is measured at a single frequency, f₀, and compared to the contralateral side, Zⱼ′ to yield an index of edema for the joint quantified at that frequency: Eⱼ=ΔZⱼ=Zⱼ−Zⱼ′|_{f}_{₀}. A major limitation of this approach is the requirement that the electrodes must be positioned identically on both the injured and contralateral joints, and both limbs should be in the same position for the measurement. Moreover, any subject with an injury on both sides of the body would not be able to employ the method. Finally, the technique requires taking simultaneous measurements from both joints, making the hardware more cumbersome than measurement on one side only.

### B. Novel Differential Measurement Technique

Extracellular fluid associated with ankle edema is delocalized and therefore free to move around in the joint space as a subject changes the position of his/her ankle. For example, as the person rotates his/her ankle, the edema moves around inside the joint space due to gravity and forces exerted by the structures inside and around the ankle. Electrodes positioned proximally and distally to the joint for BIS measurement allow current to be injected into the ankle, where different frequency components of this current travel at different depths within the tissue. Skin effect theory states that low frequency current flows deeper into the tissue, since it cannot penetrate cell membranes and must travel along extracellular paths (i.e. interstitial fluid and blood). In contrast, higher frequency current can penetrate cells, so it travels along a more superficial, shorter path between the electrodes.

Our differential measurement technique exploits this frequency-dependency of the penetration depth of current within the ankle: by moving the ankle through multiple positions during the BIS measurement, we deliberately cause some of the edematous fluid volume to move to different depths within the ankle. Low frequency currents that penetrate deeply within the tissue are dependent on the extracellular fluid for passage. A change in the volume of extracellular fluid deeper into the tissue, close to the current's path, would have a significant impact on bioimpedance measurement at low frequency. For high frequency currents that penetrate the cell membranes, they are more dependent on intracellular fluids for passage. Thus, a change in the volume of extracellular fluid close to its path would have less of an impact on the bioimpedance measurement at a high frequency. For a healthy joint, as a person moves his/her ankle there is a similar change in bioimpedance measured at high and low frequencies because, in comparison to an injured joint, there is a relatively small volume of extracellular fluid that is displaced with ankle movement. Our method compares the range of change in bioimpedance at low frequency associated with ankle position change to the range of change in bioimpedance at high frequency associated with these changes. Specifically, we compute the ratio of the changes in bioimpedance at 100 kHz to the changes in bioimpedance 5 kHz to normalize the change to a score, h_{α}, that can be relatable to all subjects:

$${h}_{α} = \frac{Δ {R}_{100 k H z}}{Δ {R}_{5 k H z}} \qquad ( 6 )$$

$$where$$

$$Δ {R}_{f} = max ( {R}_{f} ) - min ( {R}_{f} )$$

Our method includes: (1) we are performing it for localized bioimpedance measurements and (2) importantly, our h_{α} is calculated in a dynamic manner by calculating the ratio of the range of change in impedance due to positional changes, at different frequencies. Note that while this score is computed from a ratio, it is itself an absolute measure from which a particular subject can potentially be compared against a population norm. For healthy subjects we expect a score close to 1.0 and for injured subjects we expect a score that is lower (e.g., 0.5). This new method overcomes the challenge faced by electrode positioning as it depends on the differential bioimpedance measurement and not the static bioimpedance measurement. It also can include tracking the edema volume in the joint without interfering with the patient's daily functions and tasks as it uses the joint movement for evaluation.

### C. Evaluation of the System and Differential Measurement Technique in Human Subjects

### 1) Subjects and Data Collection

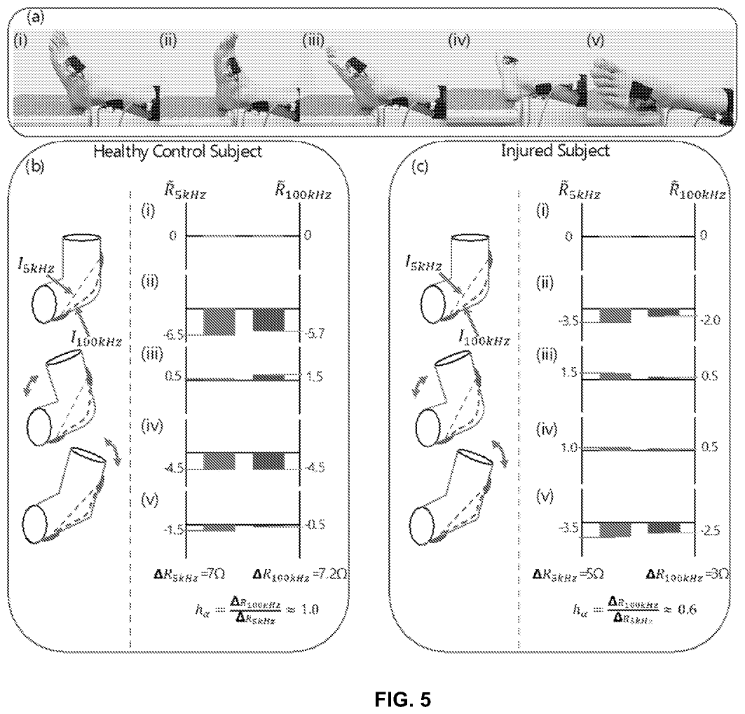

To validate the presented method, a study was performed on eight subjects, five male subjects and three female subjects between the age of 18 and 30 years old, with no recent ankle injuries and five subjects, four male subjects and one female subject between the age 18 and 30 years old with recently incurred (within two days) acute lateral ankle sprains. The study was approved by the Georgia Institute of Technology Institutional Review Board (IRB). For each subject, data were collected with the BIS system presented in this paper for 10 minutes at 5 different ankle positions composing the entire range of motion of the ankle in four axes (resting position, dorsiflexion, plantarflexion, eversion and inversion) for 2 minutes each as shown in FIG. 5(a). FIG. 5(a) provides the five different postures used in the experiment. The subjects were asked to sit upright with their legs resting horizontally at 90 degrees to their torso, to avoid a change in impedance due to blood pooling.

The protocol was performed on the eight control subjects with no recent ankle injuries five times for each ankle over a period of 21 days. For the injured subjects, only a single recording was obtained immediately following the injury (i.e., within two days).

2) Results and Discussion from Human Subject Studies

We focused on comparing the inter-subject and intra-subject variability for the two edema assessment methods mentioned in this paper—specifically, the method in which the injured side is compared against the contralateral side, and the novel differential measurement technique presented here. We also checked the capability of the new methods to differentiate healthy controls from injured joints by comparing their variance. The data collected were processed offline using Python. A 5-point FIR filter was applied to the resistance measured at 5 kHz and 100 kHz where h_{α} and Eⱼ are calculated for all subjects. Eⱼ is calculated by taking the difference in the resistance measured at 5 kHz between the left and right or right and left ankles while the subject is at resting position.

To better understand h_{α}, FIGS. 5(b) and 5(c) show the change in resistance measured at 5 kHz and 100 kHz normalized to the resting position (i.e. the change in impedance from resting position) for a healthy control subject and an injured subject respectively. FIGS. 5(b) and 5(c) also show a sketch showing the edema moving through the different current bands for (b) a healthy control subject and (c) an injured subject. For the control subject, we saw a similar range of change in resistance measured at 5 kHz and 100 kHz over the five different ankle positions. The h_{α} score for the control subject's ankle was very close to 1.0 due to the lack of extracellular edema in the ankle joint. For the injured subject, we see a similar trend for the resistance measured at 5 kHz and at 100 kHz, but the magnitude of the change in resistance is higher for the resistance measurement at 5 kHz. The h_{α} for injured ankle was 0.6 due to the existence of extracellular edema in the ankle joint which causes a bigger range of change in the resistance measured at low frequencies than at higher frequencies. Note that for the healthy subject's data presented in FIG. 5(b) the highest and lowest impedances are at positions (III) and (II), respectively, compared to positions (III) and (V) for the injured subject's data presented in FIG. 5(c). This was a common trend in the data from the human study for the healthy and the injured cases, but not conclusive to choose theses specific positions for analysis. The change in impedance at different positions suggests that the compression and relaxation of the muscles and tendons in the ankle joint have an effect on the bioimpedance measured but this change is very similar at low and high frequencies for healthy ankle joints. Hence our method compares the range of change (ΔR_{f}) in the ankle impedance at 5 kHz and 100 kHz caused by changing the ankle position in four axes independent of the position. We hypothesize that the existence of edema that is free to move in the ankle joint structure would amplify the range of change in impedance at lower frequency more than at higher frequency.

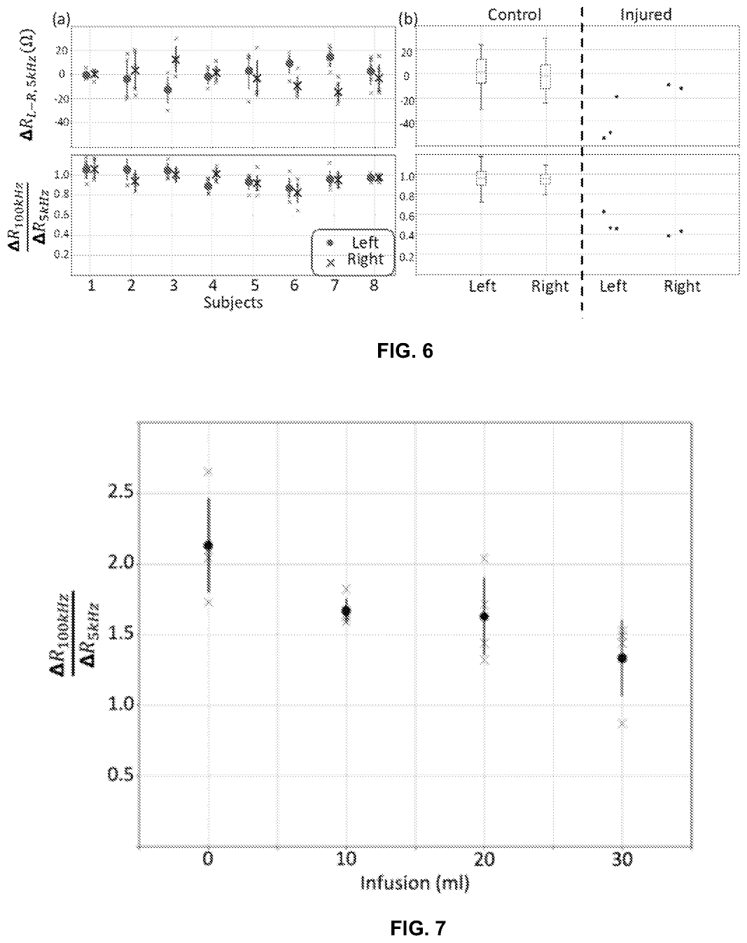

Evaluating Intra-Subject Variability: To quantify the ability of the different methods for tracking edema longitudinally, we focused on investigating intra-subject variability. Intra-subject variability is critical in determining longitudinal variation in the signal caused by day-to-day tasks performed by the subject, and thereby tracking edema longitudinally. Specifically, we compiled data from all control subject and plotted it in FIG. 6(a) showing the variation in h_{α} and Eⱼ over the duration of the study. FIG. 6(a) provides a scatter plot showing the intra-subject variability of two methods discussed in this disclosure for all control subjects over the length of this study. For Eⱼ and h_{α} we calculated the variance and standard deviation using Python. We calculated the variance of Eⱼ and h_{α} for a randomly selected subject to be 68 and 0.008, respectively, and the standard deviation was 8 and 0.1 respectively. Moreover, since Eⱼ was calculated while both ankles were in the resting position, we decided to inspect the effect of changing the ankle position on Eⱼ by calculating the average change in resistance due to the subject changing their ankle position. This came out to be 10.5Ω which is higher than the standard deviation for Eⱼ demonstrating the ineffectiveness of Eⱼ in tracking ankle joint edema if both ankles are not in the same position. Reflecting on those numbers, we observe a very small intra-subject variability for h_{α} compared to Eⱼ. We also observe an h_{α} score that is close to 1.0 for both ankles, supporting our hypothesis about h_{α} for ankles with no recent record of injury.

Evaluating Inter-Subject variability: To assess the capability of the methods to detect edema in the ankle joint, we investigated their inter-subject variability for control and injured subjects. Inter-subject variability informs us about the method's accuracy in differentiating between the injured from the control subjects. We combined Eⱼ and h_{α} from all subjects' data for the duration of the study and plotted them in FIG. 6(b). FIG. 6(b) provides a box plot showing the inter-subject variability and the separation for two methods discussed in the disclosure over the length of the study. Note that in (a) and (b), Eⱼ for the Right is the negative of Eⱼ for the Left. For control subjects, the variance for Eⱼ and h_{α} is 237 and 0.01, respectively, and the standard deviation is 15 and 0.1 respectively. For injured subjects, the variance for Eⱼ and h_{α} is 234 and 0.006, respectively, and the standard deviation is 16 and 0.08 respectively. The average of h_{α} for the control subjects was 0.955 compared to 0.5 for the injured subjects where the difference between those averages is 50 times larger than the variance of h_{α}. Also, note in FIG. 6(b) the separation between the control and injured population for h_{α} and Eⱼ where there is some overlap for Eⱼ between the control and injured population compared to none for h_{α}.

### D. Evaluation of the System and Differential Measurement Technique in Cadaver Models

### 1) Cadaver Models and Experiment Setup

To further investigate the new method's capability to track changes in edema volume in the ankle joint, we performed a study on four cadaver limbs. After the limbs were fully thawed and pre-conditioned with 5 minutes of continuous motion through the full range of motion, the ankles were manually positioned into the same five postures in FIG. 5(a) by a trained researcher. The protocol in FIG. 5(a) was repeated four times; each time 10 mL of saline solution was injected into the lateral malleolus region. Data were collected from the cadaver model ankles at 0, 10, 20 and 30 mL of infused saline using the device presented. The volume of saline injected was chosen as mentioned for the following two reasons: (1) in literature, the volume of static liquid in a healthy ankle joint was found to be between 0.13 mL and 3.5 mL and the volume of edema due to ankle sprain was found to be around 77 mL and 82 mL. Hence, injecting saline solution into the ankle joint at 10 mL increments, shows great confidence in the resolution of the method proposed in this paper. (2) From our experiments, we were able to detect swelling visually at around 30 mL of saline infusion which increases the clinical significance of the device and method.

2) Results and Discussion from Cadaver Study

Due to the significant number of dead cells, and their inability to regenerate, utilizing the cadaver models resulted in a skew for baseline h_{α}. FIG. 7 shows the reduction in the mean of h_{α} for the four cadaver models with increasing the saline injections simulating an increase in extracellular edema volume in the ankle joint. FIG. 7 provides a plot of the ratio of the resistance at 100 kHz to 5 kHz vs infusion for cadaver models. We performed statistical analysis using repeated measures ANOVA using SPSS to check the method's ability in differentiating between the four different groups which would translate to the ability to detect changes in edema volume in the ankle joint. The data passed the sphericity test where the repeated measurement ANOVA p-value was 0.004. We also performed a Least Significant Difference (LSD) post hoc test and the pairwise comparison result showed an adjusted for multiple comparisons statistical significance between 0 and 20 mL saline infused of 0.004 possibly indicating a resolution of 20 mL. This supports the proposed method's ability to not only detect extracellular edema in the ankle joint, but also track it, which is imperative for injury evaluation.

### CONCLUSION

This disclosure presents a small form-factor bioimpedance spectroscopy (BIS) system which uses a state-of-the-art calibration method based on machine learning algorithms to allow a reduction in the hardware complexity without compromising accuracy. We also presented a robust differential method to detect and track extracellular edema in the ankle joint longitudinally. We performed a study on healthy control subjects with no recent record of injury and subjects with recent ankle injuries (within 2 days) as well as cadaver models. We evaluated the method by comparing its inter-subject and intra subject variability to conventional EBI based methods of edema detection. The new method showed high accuracy in detecting and tracking extracellular edema in the ankle joint with very small variability.

### APPENDIX I

In this section we present the justification behind the calibration algorithm represented by equations (2) and (4) and compare it to the calibration procedure suggested in the datasheet. Let i_{body}(t) which is the current injected to the body at a frequency f₀ be:

i_{body}(t)=Iₒ cos(2πfₒt+ϕ) (7)

We can express i_{body}(t) in phasor form, Ĩ_{body}, as:

Ĩ_{body}=Iₒe^{jϕ} (8)

The voltage signal vᵢₙ(t), is the amplified version of the voltage drop across the load between the voltage electrodes (this load is Z=R+jX). The phasor form of this signal \tilde{V}ᵢₙ can be expressed as follows due to Ohm's law:

\tilde{V}ᵢₙ=Ĩ_{body}Z (9)

The output measurements of the AD5933 are the real and imaginary part of this phasor: re and im. The goal of calibration is to relate these to R and X. Expanding (9) allows us to come up with a new calibration algorithm. Specifically, substituting (8) into (9) results in:

$${\tilde{V}}_{i n} = {I}_{o} Z {e}^{j ϕ} = {I}_{o} \vert Z \vert {e}^{j ∡ Z} {e}^{j ϕ} = {I}_{o} \vert Z \vert cos ( ϕ + ∡ Z ) + {jI}_{o} \vert Z \vert sin ( ϕ + ∡ Z )$$

using the trigonometric addition formulas, this becomes:

\tilde{V}ᵢₙ=Iₒ|Z| cos( [symbol] Z)cos(ϕ)−Iₒ|Z| sin( [symbol] Z)sin(ϕ)+jIₒ|Z| sin( [symbol] Z)cos(ϕ)+jIₒ|Z| cos( [symbol] Z)sin(ϕ)

Now using the fact that:
R=|Z| cos( [symbol] Z) and X=sin( [symbol] Z), we have:

\tilde{V}ᵢₙ=Iₒ cos(ϕ)R−Iₒ sin(ϕ)X+jIₒ cos(ϕ)X+jIₒ sin(ϕ)R (10)

Since \tilde{V}ᵢₙ=re+j im, (8) reveals that:

re=Iₒ cos(ϕ)R−Iₒ sin(ϕ)X (11)

im=Iₒ sin(ϕ)R+Iₒ cos(ϕ)X (12)

Reorganizing (9) and (10) gives:

$$R = \frac{cos ϕ}{{I}_{o}} re + \frac{sin ϕ}{{I}_{o}} im \qquad ( 13 )$$

$$X = - \frac{sin ϕ}{{I}_{o}} re + \frac{cos ϕ}{{I}_{o}} im \qquad ( 14 )$$

Therefore both R and X can be expressed as a linear combination of re and im. Due to non-idealities in the device, constant intercept terms can be added to (12) and (13) resulting in the linear relationships:

R=α_{R}re+β_{R}im+γ_{R} (15)

X=α_{X}re+β_{X}im+γ_{X} (16)

From (13), (14), (15) and (16), α_{R} is the same as β_{X} and β_{R} is the same as −α_{X}, but in practice, after training the models in (15) and (16), we found their values to be very similar but not equal. This is largely due to the difference in the range of R and X over which the model is trained. We have experimented with another model that would ensure equal values as follows:

$$[ \begin{matrix}\begin{matrix}\begin{matrix}\begin{matrix}\begin{matrix}{R}_{1} \\ ⋮\end{matrix} \\ {R}_{N}\end{matrix} \\ {X}_{, 1}\end{matrix} \\ ⋮\end{matrix} \\ {X}_{N}\end{matrix} ] = [ \begin{matrix}{re}_{, 1} & {im}_{1} & 1 & 0 \\ ⋮ & ⋮ & ⋮ & 0 \\ {re}_{N} & {im}_{N} & 1 & 0 \\ - {im}_{1} & {re}_{1} & 0 & 1 \\ ⋮ & ⋮ & ⋮ & ⋮ \\ - {im}_{N} & {re}_{N} & 0 & 1\end{matrix} ] ⋆ [ \begin{matrix}\begin{matrix}\begin{matrix}α \\ β\end{matrix} \\ {C}_{R}\end{matrix} \\ {C}_{X}\end{matrix} ]$$

Using this model, the measurement error increased by 0.3Ω. Accordingly, we used the model above and did not assume these parameters to be necessarily equal.

### APPENDIX II

The following tables are the 2R1C impedance values used for calibration and characterization.

TABLE III
Calibration Impedances

| Re(Ω) | Ri(Ω) | C(nF) |
|---|---|---|
| 10 | 10 | 1 |
| 10 | 10 | 45 |
| 10 | 555 | 1 |
| 10 | 555 | 45 |
| 180 | 10 | 1 |
| 180 | 10 | 45 |
| 180 | 555 | 1 |
| 180 | 555 | 45 |

TABLE IV
Characterization Impedances

| Re(Ω) | Ri(Ω) | C(nF) |
|---|---|---|
| 150 | 330 | 7 |
| 150 | 180 | 10 |
| 150 | 100 | 22 |
| 120 | 150 | 15 |
| 180 | 555 | 4.7 |

This disclosure also presents a robust methodology for evaluating ankle health during ambulation using a wearable device. Methods: We developed a novel data capture system that leverages changes within the ankle during ambulation for real-time tracking of bioimpedance. The novel analysis compares the range of reactance at 5 kHz to the range of reactance at 100 kHz; which removes the reliance on a known baseline. To aid in interpretation of the measurements, we developed a quantitative simulation model based on a literature review of the effects on joint bioimpedance of variations in edematous fluid volume, muscle fiber tears, and blood flow changes. Results: The results of the simulation predicted a significant difference in the ratio of the range of the reactance from 5 kHz to 100 kHz between the healthy and injured ankles. These results were validated in 15 subjects—with 11 healthy ankles and 7 injured ankles measured. The injured subjects had lateral ankle sprains 2-4 weeks prior to the measurement. The analysis technique differentiated between the healthy and the injured population (p<<0.01), and a correlation (R=0.8) with a static protocol previously validated for its sensitivity to edema. Conclusion: The technology presented can detect variations in ankle edema and structural integrity of ankles, and thus could provide valuable feedback to clinicians and patients during the rehabilitation of an ankle injury. Significance: This technology could lead to better-informed decision making regarding a patient's readiness to return to activity and/or tailoring rehabilitation activities to an individual's changing needs.

### Systems and Experimental Design

### A. Signal Analysis

The Fricke-Morse circuit model has three components—Rₑ, Rᵢ, and Cₓ—which together describe the bioimpedance of tissue. Estimating these values requires impedance measurements at multiple frequencies and a non-linear least squares-based algorithm. Substantial measurement time and computational power are required—both of which are unfavorable for implementation of BIA in a wearable system designed to provide real-time feedback to the user. To circumvent the requirements presented in the Fricke-Morse model estimation, we devised a simple and robust method for assessing the underlying biological phenomenon within the ankle. Our method compares the changes in the reactance at two distinct frequencies recorded while the subject performs a task that stresses the joint.

### 1) The Reactance of the Tissue

This disclosure includes using the Fricke-Morse circuit model to simulate the effects of edema, collagen fiber tears and blood flow on the reactance of the tissue. The model uses resistive and capacitive values for the Fricke-Morse circuit components in the ankle joint's impedance space from the literature and our previous work as a baseline.

2) The ratio of X_{5 kHz} to X_{100 kHz}

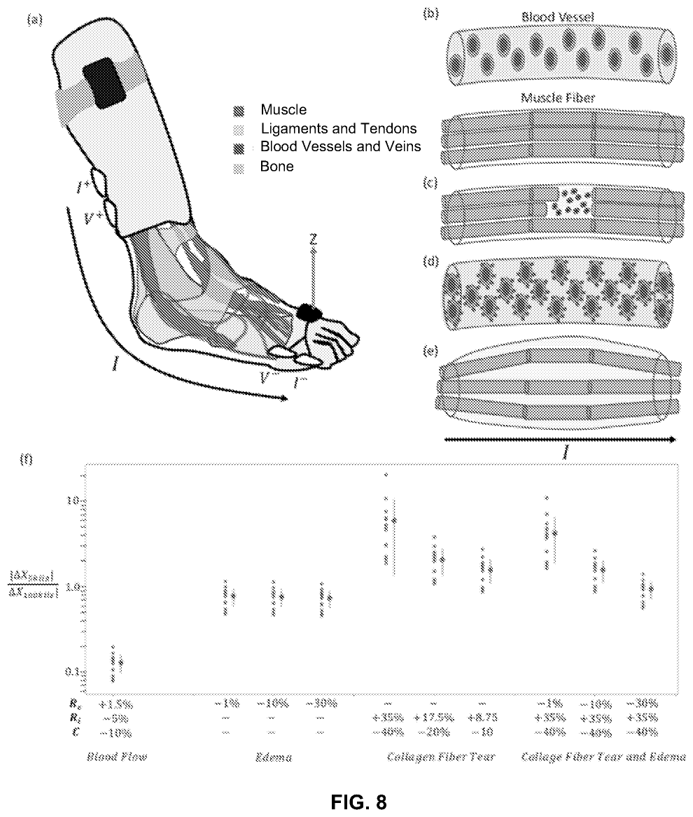

Using that model, two frequencies of electrical current are used to measure BIA. As mentioned, the low-frequency, 5 kHz current will primarily travel through the extracellular space since it is unable to pass through cellular membranes, and the high-frequency, 100 kHz current will take a more direct path using both intracellular and extracellular pathways. When these currents are applied to the ankle, they primarily flow through muscle fibers and blood vessels since the bones have low conductance and there is minimal fatty tissue and static extracellular fluid. This disclosure presented key parameters for modeling current flow. Using that research, we can simulate the effects of blood flow, edema, and collagen fiber tears on the reactance of the ankle joint. In a normal physiologic state during sustained activity, blood flow to the region increases to meet the increasing metabolic demand of the muscles (FIG. 8(d)). FIG. 8(d) shows an increase in the red blood cell count and glucose due to sustained muscle activity. When the ankle is sprained, collagen fibers may tear and edema increases (as depicted in FIGS. 8(c) and 8(e), respectively). FIG. 8(c) shows a muscle fiber tear, showing the migration of intracellular fluids to the extracellular space surrounding. FIG. 8(e) shows an increase in edema due to muscle inflammation.

In an uninjured state, when blood flow is augmented during sustained activity, the increased number of red blood cells increases the number of intracellular pathways for current to travel through, which then decreases Rᵢ. This change also increases the extracellular resistance and decreases the tissue capacitance. Additionally, the effect of changes in blood viscosity on the Fricke-Morse can be around 1.5% in Rₑ, 5% in Rᵢ and 10% in Cₓ.

In an injured state, tenocytes tear, and concomitant collagen fibers rupture, spilling intracellular contents into the extracellular space. This decreases Rₑ (since there is now more extracellular fluid) and increases Rᵢ (since there are fewer pathways through intact cells). Intact myocyte membranes can maintain the system's capacitance, therefore torn muscle fibers will also lead to a drop in Cₓ. With injury there will also be an increase in extracellular edema, which further decreases Rₑ since there is an increased volume of the conductive extracellular fluid. In their study, they reported a 30% decrease in Rₑ, 35% increase in Rᵢ and a 40% decrease in Cₓ 24 hours after the injury.

### 3) Simulation Model

To study the effect of edema, collagen fiber tear and blood flow on the localized reactance of the ankle at 5 kHz and 100 kHz, we devised a numerical analysis simulation model. The model utilizes the following equation to calculate the impedance of the Fricke-Morse model at a specific frequency (ω).

$$R + j X = \frac{{R}_{e} ( {R}_{i} + \frac{1}{j ω {C}_{x}} )}{{R}_{e} + {R}_{i} + \frac{1}{j ω {C}_{x}}} \qquad ( 17 )$$

The simulation model outputs the ratio of the changes in the reactance at 5 kHz and 100 kHz due to changes in the Fricke-Morse components from baseline. The baseline impedances used are the bioimpedance spectroscopy data collected from our previous study. Specifically, we used non-linear least squares to estimate the values of the Fricke-Morse circuit components from the bioimpedance spectroscopy measurements of 14 healthy ankles. The estimated Fricke-Morse parameters are presented in Appendix III. For each baseline ankle joint bioimpedance, we simulated the effects of blood flow, edema, collagen fiber tear and collagen fiber tear accompanied with edema on the ankle joint using percentage changes reported in the literature for each of these phenomena as mentioned earlier and shown in FIG. 8(f). FIG. 8(f) provides logarithmic scale of the ratio of the change in the low frequency reactance to the high frequency reactance due simulating the effect of blood flow, edema, collagen fiber tear, and collagen fiber tear accompanied with edema in the ankle joint on the baseline ankle impedance of 14 healthy subjects.

In FIG. 8(f), we present the results of our simulation of BIA in the ankle. When comparing the ratio of change in reactance at 5 kHz and at 100 kHz we found a significant difference between healthy and injured ankles as shown in FIG. 8(f). This result is consistent with our expectations based on the impact of the described pathophysiologic changes during activity and injury. The findings of this simulation encourage further research and hardware development into the BIA phenomenon and its clinical uses.

### 4) Limitations of Simulation Model

In FIG. 8(f), we also present the effect of varying the intensity in the changes in the Fricke-Morse parameters due to edema, collagen fiber tear, and edema and collagen fiber tear combined. In the case of increasing edema, reducing Re further led to a change in the reactance at 5 kHz and 100 kHz at a similar rate making the ratio of the changes in the reactance at 5 kHz to the change in the reactance at 100 kHz a viable method for the detection of edema, but not quantifying it.

### 5) Differentiating Between Edema and Muscle Tear

After the acute phase of an injury, the tissues enter a phase of rebuilding which includes a reduction in edema and increasing collagen fiber strength. The level of edema and strength of the reforming fiber are indicative of the progress of the rehabilitation and the probability of reinjury. In FIG. 8(f), the ratio of the change in the reactance at 5 kHz versus at 100 kHz successfully differentiates healthy from injured ankles, but differentiating swelling from collagen fiber strength is a different challenge. In our previous work, we studied the effect that changes in ankle position have on low (5 kHz) and high-frequency (100 kHz) resistance measurements. We showed that these positional changes move the extracellular fluids around the joint altering the extracellular resistance (Re) which in turn predominantly impacts the low-frequency resistance (extracellular fluid dependent) compared to the high-frequency resistance. Although, in this example we are using the changes in the reactance, instead of the resistance, at low and high frequency for our analysis, the concept of extracellular fluids shifting in the tissue due to the pressure from the joint structure caused by its rotation still applies here. Alternatively, or in addition, changes in the resistance at low and high frequency can be used for analysis.

In this work, we measure the bioimpedance of the ankle joint while subjects are walking using our wearable sensing hardware. Normally, the instantaneous changes observed in the ankle's bioimpedance during a subject's gait cycle are possibly due to the tendons and ligaments, and varying blood flow in the current path. However, if there exists edematous extracellular fluid, the pressure from the joint structure would shift the fluid around it changing the extracellular resistance (Rₑ) in the current path. We hypothesize that in the presence of joint edema, there will be an increase in the instantaneous change per step in the low frequency reactance measurements compared to the lower frequency measurements as shown in FIG. 8(f). Nevertheless, these positional changes should not compound throughout the duration of a walking session. Rather, we hypothesize that the baseline changes in bioimpedance from the beginning of a walking session are mainly due to micro-damage to any recently injured tendons, ligaments, and tissue coupled with low-level edema. To test these hypotheses, we developed two metrics for comparing the range of the reactance at 5 kHz to the range of the reactance at 100 kHz: (1) per step (ĥ_{α}), and (2) per walking session (>200 steps) (β)

### B. Software Model Development

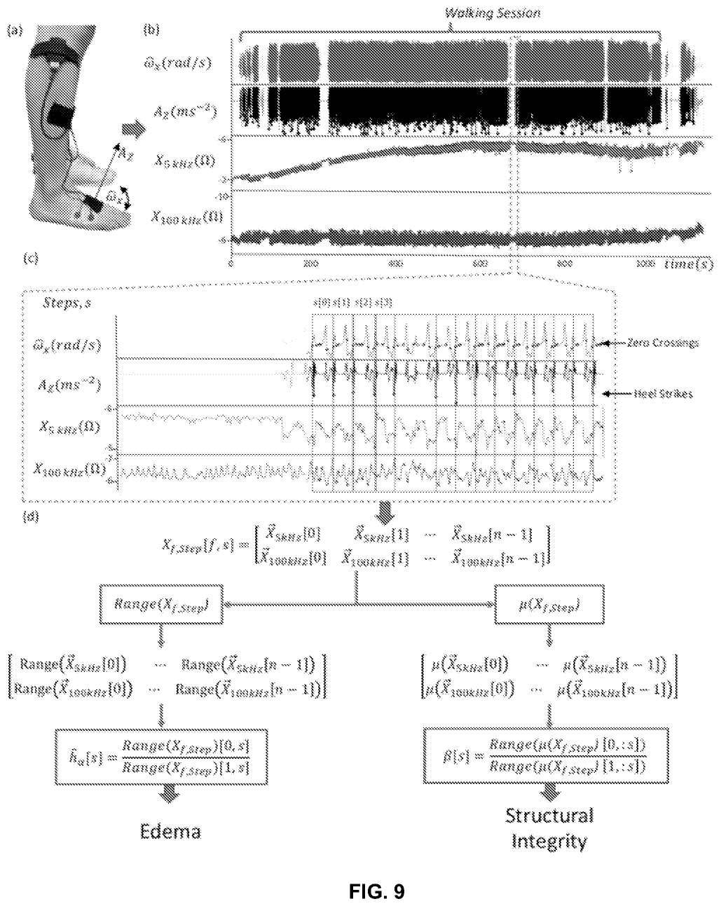

To compute ĥ_{α} and β, it is necessary to split the reactance measured at the two frequencies based on each subjects' steps as shown in FIG. 9. FIG. 9 provides date analysis workflow for determining presence of edema and disruption to structural integrity to the ankle. FIG. 9(a) provides the data acquisition system placed on the subject's leg with the necessary current and voltage electrodes placed distally and proximally to the ankle joint and the IMU placed on the foot. FIG. 9(b) provides sample data of a representative injured subject's X-axis angular velocity, Z-axis acceleration, and reactance measured at 5 kHz and 100 kHz. FIG. 9(c) provides a magnified view of the sample data showing how the data windows are created and used in splitting to split the reactance data into vectors per step. FIG. 9(d) provides the reactance vectors per step are used in the model to detect edema and collagen fiber tear in the ankle joint. This windowing uses the inertial measurement unit (IMU) employed on our custom hardware. The IMU captures the angular velocity of the foot, which is used to determine if the subject is moving. This is performed by taking 3-second windows of the angular velocity and convolving those values on themselves to compute the energy of that window's angular velocity (ω[t]) as shown in the following equation.

$${E}_{ω} [ t ] = {\sum }_{t = n}^{n + 3} {\vert ω [ t ] \vert}^{2} ( rad / {s}^{2} ) \qquad ( 18 )$$

That energy is compared against an experimentally determined threshold of 10,000. If the energy is higher than that threshold, the peaks of the Z-axis (lateral) acceleration signal from the IMU are used to identify the heel-strikes which mark the beginning of each step as shown in FIG. 9(c). Each peak needs to be at least 350 ms from the previous peak and beyond a certain threshold (1 g) to remove errors from irregularities in the signal. Since the bioimpedance is sampled at a lower frequency than the IMU, the start and end of each step's bioimpedance window is identified by finding the absolute minimum time difference between the time of the heel strike and the time of the bioimpedance measurements. These data are then used in the model presented in FIG. 9(d).

After splitting the reactance measured to per step arrays as shown in FIG. 9(d), the range and mean of each step's reactance is calculated. ĥ_{α} is calculated by taking the ratio of the range per step of the reactance at 5 kHz to the range per step of the reactance at 100 kHz. To calculate β at any step (s), the range of the mean of the reactance per step from the start of the walking session to step s is calculated. The ratio of this range at 5 kHz to 100 kHz is then taken to calculate β as shown in FIG. 9(d).

### C. Hardware and Firmware

To detect edema and structural damage in the ankle joint while the subject is performing their daily activities, we modified our previous hardware and firmware. For the hardware, as discussed, adding an IMU to the peripheral cable allows position and movement of the limb to be measured during the gait cycle. The IMU LSM6DS3 (ST, Geneva, Switzerland) was selected for its accuracy, low power consumption and ease-of-interfacing. For the firmware, an interrupt-based architecture was implemented where the sensors' (Bioimpedance and IMU) state machines are updated every 1 ms and the data are saved onto a double-buffer to ensure a constant sampling rate from the sensors. The buffered data are saved onto an SD-card which is processed offline using Python. Relevant system properties are shown in Table V.

TABLE V
System Properties

| Parameter | Value | |
|---|---|---|
| Battery life | 12 | hours |
| Size | 5.2 × 3.8 × 1.8 | cm |
| Weight | 32 | g |
| Bioimpedance: | | |
| Frequencies: | 5 kHz, 100 kHz | |
| Sampling Rate | 10 | Hz |
| Resolution | 0.2 | Ω |
| Inertial Measurement Unit | | |
| Number of Axis | 6 | |
| Sampling Rate | 50 | Hz |

### D. Data Collection Protocol for Method's Evaluation

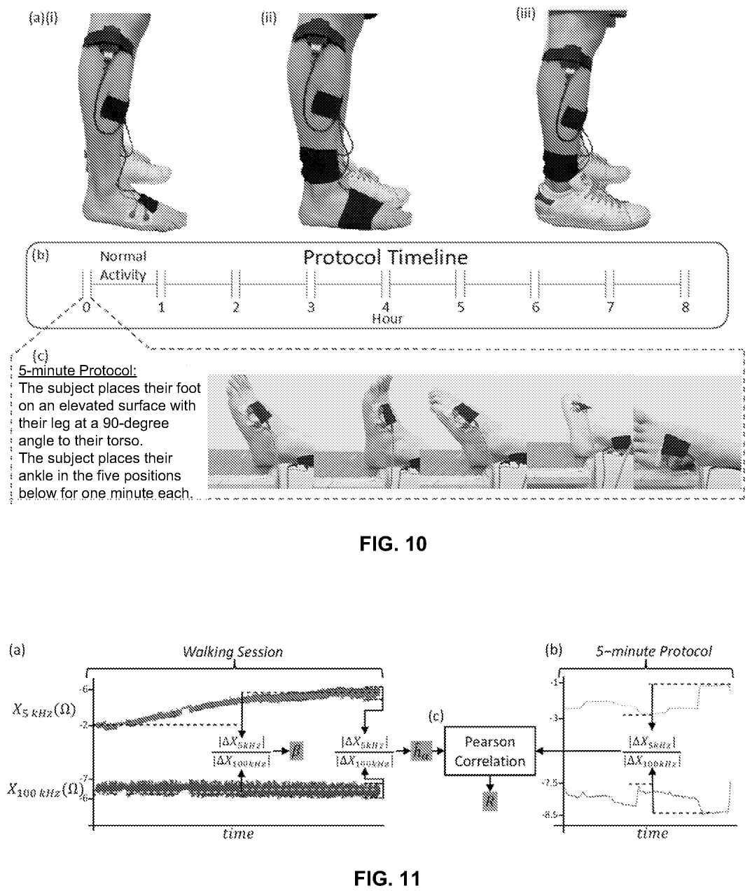

To validate the hypothesis that ĥ_{α} can detect edema in the ankle, we recorded data from 15 subjects during ambulation and asked them to perform the BIA ankle positional protocol as described in this disclosure and depicted in FIG. 10(c) once per hour. The data collection protocol was approved by the Georgia Institute of Technology Institutional Review Board, and all subjects provided written informed consent before participating in the study. This positional protocol was previously shown to correlate with edema. In this study, we sought to determine if ĥ_{α} measured during walking also correlated with edema. We also sought to test if β (the difference in impedance after a continuous walking session) can differentiate between healthy and injured populations.

FIG. 10 depicts the overall testing protocol. FIG. 10 provides a recording setup and 8-hour recording protocol timeline. FIG. 10(a) shows the wearable data acquisition is placed on the subject's leg. FIG. 10(b) shows that the overall recording protocol took 8 hours with the 5-minute positional protocol as depicted in FIG. 10(c) being performed every hour. The modified system is placed on the subject's ankle as shown in FIG. 10(a). Red dot gel electrodes (3M, Saint Paul, Minn.) are used for bioimpedance measurements. The electrode snaps and IMU are secured using Kinesio tape (Kinesio, Albuquerque, N. Mex.) to further secure them and dampen the forces from movement. With the recording setup in place, the subjects performed the 5-minute positional protocol shown in FIG. 10(c). The subjects then perform their normal daily activities while performing the 5-minute positional protocol every hour as depicted in FIG. 10(b).

The participants were recruited via word of mouth by either the engineering research staff or the Georgia Institute of Technology athletic trainer of the study team. The sensors were outfitted in the lab or the athletic center in the early morning to reduce any residual effect from prior movement on the data. The subjects were then instructed to go about their daily activities for eight hours. After the eight hours of data collection, the subjects returned to the lab or the athletic center for the device to be removed.

The study was performed on 15 subjects between the ages of 18 and 30. Of these subjects, 7 had an ankle injury in the two to four weeks prior to data collection. Data from the contralateral healthy ankle were also collected from 3 of the 7 injured subjects on a separate day. On the remaining 8 healthy subjects, data were collected from their dominant ankle due to its higher chance of injury. In total, 11 healthy ankles and 7 injured ankles were recorded. Four of the injured ankles were diagnosed by a medical professional and the rest were self-reported. The injuries were grades 1-2 lateral ankle sprains. The data were analyzed offline using Python.

### E. Statistical Analysis

To test the ability of the methods presented to separate between healthy and injured groups, we first tested the data for normality using Wilk-Shapiro test. Since one of the data groups (healthy ĥ_{α}) failed the normality test, we used Wilcoxon rank-sum test where a p-value less than 0.05 is considered significant. For each score (ĥ_{α} and β) Cohen's (d) effect size between the healthy and injured group was also calculated where an effective size higher than 1.4 is considered large effect. An example of the variables extracted from the model in FIG. 9 is presented in FIG. 11. FIG. 11 provides a method for comparing the full walking session to 5 minute protocol. FIG. 11(a) provides the range of change in the reactance measured at 5 kHz and 100 kHz during a continuous walking session is used to calculate β. FIG. 11(b) provides the range of the change in the reactance measured at 5 kHz and 100 kHz per step is used to calculate ĥ_{α}. FIG. 11(c) shows the mean ĥ_{α} of the last 10 steps is correlated to the ratio of the range of change in the reactance measured at 5 kHz and 100 kHz using Pearson's correlation. We also used Pearson correlation tests to show the correlation of ĥ_{α} to the static protocol.

### Results and Discussion

### A. Ratio of the Ranges of Reactance Per Step (ĥ_{α})

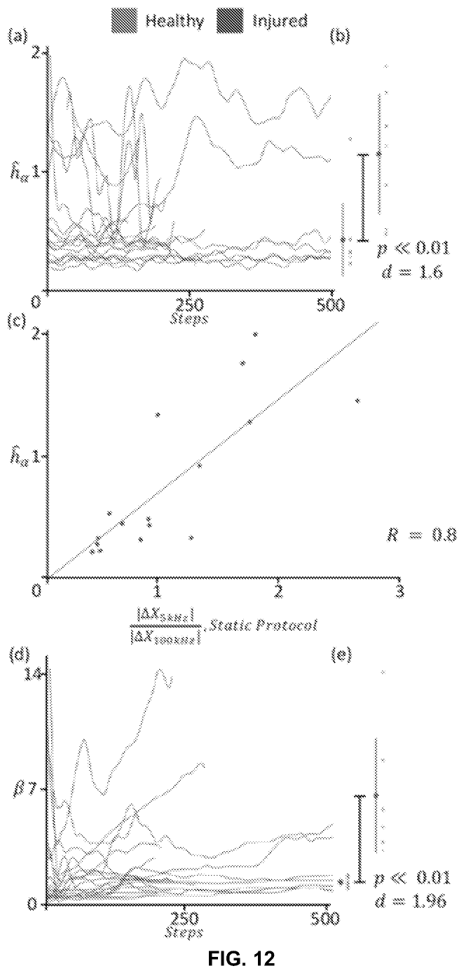

To test our hypothesis that ĥ_{α} is sensitive to detecting edema, we correlated the ratio of the range of the reactance at 5 kHz to the range at 100 kHz from FIG. 11(a) against those values found during the 5-minute, static positional protocol from FIG. 11(b) as shown in FIG. 11(c). To ensure that no residual edema or muscle tear from prior walking sessions skewed the result, data from the first substantial continuous walking session were used. In this context, a “substantial” session is considered one in which the subject walks for more than 200 steps with a maximum of pause of 1 minute between successive steps. For all 15 subjects, the mean of ĥ_{α} was calculated from the last 10 steps of the first walking session and tested for correlation with the 5-minute positional protocol performed immediately after that session for all 15 subjects as shown in FIG. 11(c). This comparison yielded a Pearson's correlation coefficient of 0.8 as shown in FIG. 12(c). This supports the hypothesis that ĥ_{α} is sensitive to edema, since the positional protocol was previously shown to correlate with edema levels. The results of ĥ_{α} and the ratio of the range of X_{5 kHz} to X_{100 kHz} from the static protocol for the injured and healthy group are presented in FIGS. 12(a)-(c) and tested for statistical significance (p=0.0021) and Cohen's effect size of 1.6. FIG. 12(a) provides a plot showing h_{α} vs steps for all subjects and FIG. 12(b) provides a scatter plot of the mean of h_{α} for the last ten steps for the healthy and injured groups showing a statistically significant p-value. FIG. 12(c) provides a plot of the mean h_{α} at the last ten steps in a continuous walking session, correlated the output of the 5-minute protocol done after with a Pearson correlation coefficient of 0.8. This indicates the method's ability to differentiate between healthy and injured ankles. There is also similarity between the injured and healthy population ĥ_{α} scores in FIG. 12(b) and the results of the simulation model for changes in edema and blood flow from FIG. 8(f).

### B. Ratio of the Ranges of Reactance Per Walking Session (β)

During a substantial period of walking, micro-tears are expected to occur in the ligaments, tendons, and connective tissue. To better understand the impact that this degradation of tissue integrity has on the reactance measured using BIA, we calculated the range of reactance recording during a continuous walking period as shown in FIG. 11(a). FIG. 11(a) provides the range of change in the reactance measured at 5 kHz and 100 kHz during a continuous walking session is used to calculate β. Significant differences in this range between the injured and healthy groups when taking the β from the last step of the continuous walking session was found (p<<0.001) and Cohen's d effect size of 1.96 as shown in FIGS. 12(d) and 12(e). FIG. 12(d) provides a plot showing β vs steps for all subjects and FIG. 12(e) provides a scatter plot of the β at the last step of a continuous walking session showing a statistically significant p-value. To control for inter-subject inter-session variability (particularly in the number of steps per walking session), the β is also calculated at the 200th step of the first substantial walking session for all subjects and a significant difference between the injured and the healthy groups was found (p<<0.01). There is also similarity between the β scores for the healthy and injured population shown in FIG. 12(e) and results of the simulation model for collagen fiber tear and blood flow from FIG. 8(f).

### C. Zero Crossing Data Analysis

During the typical gait cycle, the ankle's direction of rotation changes (thus, its angular velocity=0) four times: (1) at a neutral position (i.e., with the foot and shank at or near 90° to each other) just prior to heel-strike, (2) in a slightly plantarflexed state once the foot is flat on the ground just after heel-strike, (3) in a dorsiflexed state just prior to heel-rise, and (4) in a plantarflexed state just prior to toe-off leading into swing phase. These same joint configurations are performed in the static positional protocol, providing points of comparison between the dynamic (walking) and static (positional) tasks. The software model presented was tested using only the data closest to the zero crossings by choosing the bioimpedance measurements that had the absolute minimum time difference from the time of the zero-crossings. For the ĥ_{α} calculated using the zero crossings data, the Spearman's correlation coefficient with the ratio of the range of reactance at 5 kHz to the range of reactance at 100 kHz from the 5-minute protocol is 0.63. The calculated p-value is p<<0.01 for the separation between the healthy and injury group. The difference in the correlation score using all bioimpedance measurements and the measurements closest to the zero crossings may be due to the relatively low sampling rate of the bioimpedance or due to a delayed response for the impedance from the changes in the ankle position caused by the loading of the joint at these positions. In some cases, the nearest bioimpedance measurement to the zero crossing was up to 50 ms away. For the β score using the data closest to the zero crossings, the p-value is <<0.01.

Using only the data at the angular velocity's zero crossing would enable significant reduction in the bioimpedance samples needed and hence the power consumption, as the bioimpedance can be measured only at the zero-crossings using an interrupt-based approach. This would allow the software model to be fully implemented on an embedded processor in a wearable-form factor.

### D. Implications of Findings on Feasibility of Wearable Joint Health Monitoring

The work presented in this paper provides a robust method for capturing and analyzing bioimpedance in the ankle. Previous work in this field typically prescribed a set of controlled exercises to ensure accurate measurements. In our work, we designed a solution for adapting this technology into a wearable form factor that leveraged the impact of ambulation on the signals rather than mitigating them. Additionally, this novel method of signal interpretation requires minimal algorithmic and computational complexity making it suitable for being embedded into a miniaturized, wearable system. The results of this algorithm are presented in this disclosure and demonstrate that this technique is capable of real-time detection of edema and tissue integrity changes in the ankle during activities of daily living. This usability improvement and enhanced algorithm enables real-time, joint health status updates for the wearer. These notifications, if properly utilized could greatly aid in personalizing clinical rehabilitation efforts.

### CONCLUSION

In this disclosure, we present for the first time a system that performs BIA in the context of detecting edema and structural integrity in the ankle joint while a subject is walking. The system fuses IMU and bioimpedance signals to evaluate and detect edema and muscle tears, which are crucial for physicians to understand the recovery progress and the subject's ability to return to activity. The analysis technique for interpreting BIA while walking was first validated in a simulation model using parameters and results from the literature and a human subjects study. The simulation results held promise, so 15 subjects both with and without injury were recorded while walking. Two metrics were developed to describe the inter-step range of reactance and the intra-walking session range of reactance. Both metrics statistically separated the injured ankles from the healthy ankles (p<<0.01). The inter-step analysis was shown to correlate with edema. The intra-session range of reactance was thought to also include the effects of microtears on the tissue.

### APPENDIX III

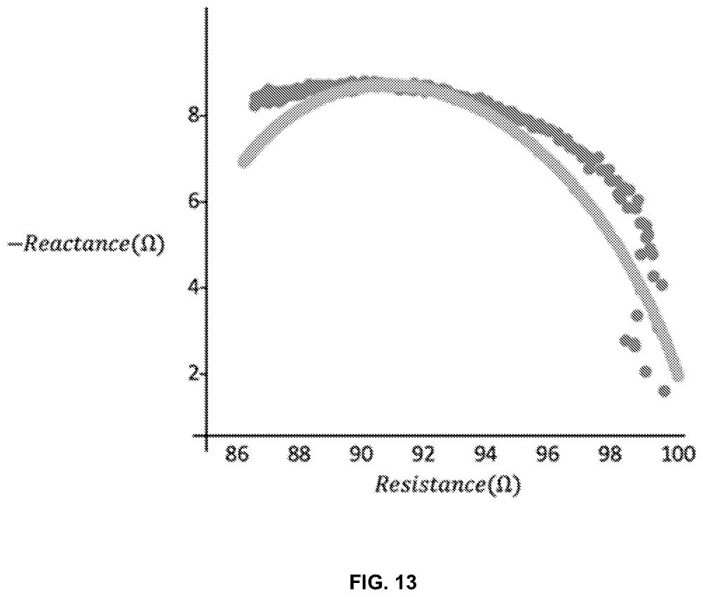

In this section we present the algorithm used to calculate the values of the Fricke-Morse components used as baseline in our simulation model. Bioimpedance spectroscopy data were collected over the frequency range from 5 kHz to 100 kHz with a resolution of 371 Hz for a total of 256 bioimpedance measurements per sweep. Using Python, we created a function that calculates real component the Fricke-Morse impedance for any given Rₑ, Rᵢ and Cₓ values at the same frequencies from our bioimpedance spectroscopy data. This function along with the data collected from the ankle joint from our previous work are inputted into SciPy's curve fit function to estimate the Fricke-Morse circuit components for each ankle. FIG. 13 shows an example bioimpedance spectroscopy of a healthy ankle joint and the estimated Fricke-Morse impedance using our algorithm. FIG. 13 provides an example of localized bioimpedance spectroscopy data and its associated Frick-Morse estimated parameters. (Rₑ˜100, Ω Rᵢ˜520Ω, Cₓ=10 nF). Table VI shows the Fricke-Morse component values for 14 healthy ankles.

TABLE VI
Ankle Fricke-Morse Circuit Component Values

| Rₑ(Ω) | Rᵢ(Ω) | Cₓ (nF) |
|---|---|---|
| 102 | 520 | 9.6 |
| 107 | 633 | 8 |
| 160 | 940 | 5.4 |
| 163 | 1178 | 3.3 |
| 115 | 543 | 9.2 |
| 108 | 770 | 5.2 |
| 113 | 877 | 5.4 |
| 152 | 676 | 7.7 |
| 127 | 746 | 6.5 |
| 100 | 869 | 5 |
| 128 | 1022 | 5.5 |
| 108 | 765 | 6 |
| 137 | 577 | 10 |
| 101 | 511 | 9.6 |

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A system for assessing joint health comprising:
a joint sensor configured to measure at least one non-acoustic characteristic of a joint;
a bioimpedance sensor configured to measure bioimpedance of the joint exposed to electrical current at a plurality of frequencies;
a processor; and
a memory, the memory comprising instructions that, when executed by the processor, cause the processor to provide an assessment of joint health through interpretation of measurements from the joint sensor and the bioimpedance sensor.

2. The system of claim 1, wherein one or more of:
the assessment of joint health can differentiate between a healthy joint and an injured joint;
the assessment of joint health occurs in real-time during movement of the joint;
the assessment of joint health comprises a detection of changes in edema in the joint during movement of the joint;
the assessment of joint health comprises a detection of changes in tissue integrity during movement of the joint;
the bioimpedance sensor is configured to sense reactance at the plurality of frequencies; and/or
the bioimpedance sensor is configured to sense resistance at the plurality of frequencies.

3.-7. (canceled)

8. The system of claim 2, wherein the memory further comprises instructions that, when executed by the processor, cause the processor to one or both:
compare changes in bioimpedance at the plurality of frequencies during movement of the joint to determine a ratio of the changes in bioimpedance; and/or
provide the assessment of joint health based at least in part on the ratio of the changes in bioimpedance.

9. (canceled)

10. The system of claim 1, wherein:
the joint sensor measures a gait cycle;
the bioimpedance sensor is configured to sense a range per step of bioimpedance at the plurality of frequencies; and
the memory further comprises instructions that, when executed by the processor, cause the processor to:
provide a window of time representing each step of the gait cycle at least in part from measuring a heel strike; and
detect changes in joint edema at least in part by taking a ratio of the range per step of bioimpedance at the plurality of frequencies.

11.-13. (canceled)

14. The system of claim 1, wherein:
the joint sensor measures a walking session;
the bioimpedance sensor is configured to sense a range per walking session of bioimpedance at the plurality of frequencies; and
the memory further comprises instructions that, when executed by the processor, cause the processor to detect changes in tissue integrity in the joint at least in part by taking a ratio of the range per walking session of bioimpedance at the plurality of frequencies.

15.-16. (canceled)

17. The system of claim 1, wherein the joint sensor is a kinematic sensor configured for sensing characteristics related to joint movement.

18. The system of claim 1, wherein the joint sensor comprises one or more inertial measurement units.

19. The system of claim 1, wherein the joint sensor is configured to measure at least angular velocity at the joint; and
wherein the bioimpedance sensor is configured to sense characteristics related to bioimpedance of the joint when the angular velocity equals zero during movement.

20. (canceled)

21. The system of claim 1, wherein the plurality of frequencies comprises a first frequency and a second frequency; and
wherein one or more of:
the bioimpedance sensor delivers a first current at the first frequency such that the first current can propagate through extra-cellular fluid;
the bioimpedance sensor delivers a second current at the second frequency such that it can propagate through intra-cellular fluid and extra-cellular fluid;
the first frequency is 1-50 kHz; and/or
the second frequency is 50-1000 kHz.

22.-25. (canceled)

26. The system of claim 1, wherein the joint sensor comprises a first wearable sensor for placement proximate the joint; and
wherein the bioimpedance sensor comprises a second wearable sensor for placement proximate the joint.

27. The system of claim 1 further comprising an output capable of providing an indication of joint health to a user of the system.

28. The system of claim 1 further comprising a wireless communicator.

29. The system of claim 1, wherein the joint is an ankle joint.

30. The system of claim 1, wherein the memory further comprises instructions that, when executed by the processor, cause the processor to perform a full frequency sweep analysis when the joint is not in movement.

31. A system for assessing ankle joint health comprising:
an ankle joint sensor configured to measure at least one non-acoustic characteristic of an ankle joint during a walking session;
a bioimpedance sensor configured to:
measure bioimpedance of the ankle joint exposed to electrical current at a plurality of frequencies; and
sense a range per walking session of bioimpedance at the plurality of frequencies;
a processor; and
a memory, the memory comprising instructions that, when executed by the processor, cause the processor to:
compare changes in bioimpedance at the plurality of frequencies during movement of the ankle joint to determine a ratio of the changes in bioimpedance;
cause the processor to detect changes in tissue integrity in the joint at least in part by taking a ratio of the range per walking session of bioimpedance at the plurality of frequencies; and
provide an assessment of ankle joint health based at least in part on the ratio of the changes in bioimpedance.

32. A method for assessing joint health comprising:
measuring, with a joint sensor, at least one non-acoustic characteristic of a joint;
measuring, with a bioimpedance sensor, bioimpedance of the joint exposed to electrical current at a plurality of frequencies; and
providing, with a memory and a processor, an assessment of joint health through interpretation of measurements from the joint sensor and the bioimpedance sensor;
wherein when the joint sensor measures a gait cycle, the method further comprises:
providing a window of time representing each step of the gait cycle at least in part from measuring a heel strike; and
detecting changes in joint edema at least in part by taking a ratio of a range per step of bioimpedance at the plurality of frequencies;
wherein the bioimpedance sensor is configured to sense the range per step of bioimpedance at the plurality of frequencies; and
wherein when the joint sensor measures a walking session, the bioimpedance sensor is configured to sense a range per walking session of bioimpedance at the plurality of frequencies, and the method further comprises detecting changes in tissue integrity in the joint at least in part by taking a ratio of the range per walking session of bioimpedance at the plurality of frequencies.

33.-59. (canceled)

60. The method of claim 32, wherein the joint is an ankle joint.

61. The method of claim 32 further comprising performing a full frequency sweep analysis when the joint is not in movement.
